(19) United States
(12) Patent Application Publication
(10) Pub. No.: US 2009/0169526 A1
(43) Pub. Date: Jul. 2, 2009

(54) BMP-6 ESTROGEN RESPONSIVE ELEMENT AND METHODS OF USE THEREOF

(75) Inventors: Peter Van Nest Bodine, Havertown, PA (US); Dan Gazit, Charlottesville, VA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: Wyeth; Yissum Research Development Company of the Hebrew University of Jerusalem

(21) Appl. No.: 12/335,251

(22) Filed: Dec. 15, 2008

Related U.S. Application Data
(62) Division of application No. 11/053,666, filed on Feb. 9, 2005.
(60) Provisional application No. 60/543,270, filed on Feb. 11, 2004.

(51) Int. Cl. A61K 48/00; A61P 19/00; C12N 15/09; C12Q 1/68

(52) U.S. Cl. 424/93.7; 514/44; 435/455; 435/6

(57) ABSTRACT

This invention is directed to a BMP-6 ERE nucleic acid sequence, vectors, cells and compositions comprising same. This invention also provides methods for identification of estrogen agonists, antagonists and therapeutic applications of same. This invention also provides methods of treating conditions, which are associated with estrogen insufficiency or with lack of response to external estrogen or agonists thereof utilizing sequences, vectors, cells and/or compositions described herein.

### CROSS REFERENCE TO RELATED APPLICATIONS

This application is a divisional of U.S. application Ser. No. 11/053,666, filed Feb. 9, 2005, which claims priority to U.S. Provisional Application No. 60/543,270, filed Feb. 11, 2004, which are hereby incorporated in their entirety.

### FIELD OF THE INVENTION

The present invention is directed to methods for regulating BMP-6 expression in mesenchymal stem cell populations, and nucleic acids, vectors, and compositions for effecting the same, and therapeutic applications arising from their utilization.

### BACKGROUND OF THE INVENTION

Throughout adult life, bone is continually remodeled through interactive cycles of bone formation and resorption (bone turnover). Bone resorption is typically rapid, and is mediated by osteoclasts (bone resorbing cells), which are cells of a mononuclear phagocytic precursor that differentiate at bone remodeling sites. Following bone resorption by osteoclasts, osteoblasts (bone forming cells) are recruited to the site, and slowly replace lost bone tissue. The various cell types that participate in bone remodeling are regulated, in turn, by interacting systemic (e.g., hormones, lymphokines, growth factors, vitamins) and local factors (e.g., cytokines, adhesion molecules, lymphokines and growth factors). The fact that completion of this process normally leads to balanced replacement and renewal of bone indicates that the molecular signals and events that influence bone remodeling are tightly controlled.

A number of bone growth disorders are known which cause an imbalance in the bone remodeling cycle. Chief among these are metabolic bone diseases, such as osteoporosis, osteoplasia (osteomalacia), chronic renal failure and hyperparathyroidism, which result in abnormal or excessive loss of bone mass (osteopenia). Other bone diseases, such as Paget's disease, also cause excessive loss of bone mass at localized sites.

Osteoporosis is a structural deterioration of the skeleton caused by loss of bone mass resulting from an imbalance in bone formation, bone resorption, or both, such that resorption dominates bone remodeling, thereby reducing the weight-bearing capacity of affected bone. In a healthy adult, the rate at which bone is formed and resorbed is tightly coordinated so as to maintain the renewal of skeletal bone. In osteoporotic individuals, however, an imbalance in bone remodeling develops, which results in loss of bone mass and in the formation of microarchitectural defects in the continuity of the skeleton. Osteoporosis affects about 50% of women, and about 10% of men, over the age of 50 in the United States. In individuals with osteoporosis, increased loss of bone mass results in fragile bones and, as a result, increased risk of bone fractures. Other bone-resorption diseases, such as Paget's disease and metastatic bone cancer, present similar symptoms.

Bone morphogenetic proteins (BMPs) are members of the transforming growth factor β (TGF-β) superfamily and originally identified by their presence in bone-inductive extracts of demineralized bone (Wozney et al., 1988; Rosen et al., 1996). It has long been suspected that the primary target cells for BMP action is an early osteoblast progenitor or the mesenchymal stem cell (Oreffo et al., 1999). Recombinant human BMP-2, a member of the BMP family, induces cartilage and bone formation in vivo (Wozney et al 1988, Wang et al 1990, Gazit et al 1999) and osteogenic differentation of several mesenchymal cell types in vitro (Katagiri et al 1990; Theis et al 1992; Wang et al 1993; Yamaguchi et al 1996; Hanada et al 1997; Gazit et al 1999; Moutsatsos et al 2001; Turgeman et al 2001).

Estrogen is one of the key steroidal hormones essential for maintenance of skeletal homeostasis, as reflected in the widespread and beneficial use of estrogen replacement therapy to combat postmenopausal osteoporosis in women. It was shown (Zhou et al 2001) that mouse bone marrow mesenchymal stem cells (MSCs) have both estrogen receptor α and β, and mouse MSCs are the anabolic targets of estrogen action.

### SUMMARY OF THE INVENTION

This invention provides BMP-6 estrogen responsive element (ERE) nucleic acids, vectors, cells, compositions and methods affecting estrogen responsive BMP-6 expression.

In one embodiment, the invention provides a method of regulating expression of BMP-6 in a subject comprising the steps of administering: (a) a stem cell comprising a BMP-6 ERE and expressing, or engineered to express, an estrogen receptor; and (b) an effective amount of an estrogen or estrogen agonist, thereby regulating expression of BMP-6 in the subject.

In one embodiment, the invention provides a method of regulating expression of BMP-6 in a subject comprising the steps of administering: (a) a vector comprising a BMP-6 ERE; and (b) an effective amount of an estrogen or estrogen agonist, thereby regulating expression of BMP-6 in the subject.

In another embodiment, this invention provides for a method of regulating expression of BMP-6 in a subject comprising administering to said subject: (a) a vector comprising a nucleic acid sequence encoding for an estrogen receptor or a functional fragment thereof, to at least one cell comprising a BMP-6 ERE operably linked to a BMP-6 gene; and (b) an effective amount of an estrogen or estrogen agonist, thereby stimulating expression of BMP-6 in said subject.

In another embodiment, this invention provides for a method of regulating expression of BMP-6 in a subject comprising administering to said subject: (a) an effective amount of a composition comprising a cell expressing an estrogen receptor or a functional fragment thereof, further comprising a BMP-6 ERE operably linked to a BMP-6 gene; and (b) an effective amount of an estrogen or estrogen agonist, thereby stimulating expression of BMP-6 in said subject.

In another embodiment, this invention provides a method of increasing the responsiveness of a cell to an estrogen or estrogen agonist comprising the step of contacting said cell with a vector comprising a BMP-6 ERE, thereby increasing the responsiveness of the cell to estrogen.

In another embodiment, this invention provides a method of enhancing bone repair in a subject in need comprising the steps of administering to said subject: (a) an isolated nucleic acid comprising a BMP-6 ERE operably linked to a BMP-6 gene; and (b) an effective amount of an estrogen or estrogen agonist; thereby enhancing repair of the bone in the body of the subject in need.

In another embodiment, this invention provides a method of enhancing bone repair in a subject in need comprising the steps of administering to said subject: (a) an effective amount of a composition comprising a cell expressing an estrogen receptor, or a functional fragment thereof, further comprising a BMP-6 ERE operably linked to a BMP-6 gene and (b) an effective amount of an estrogen or estrogen agonist; thereby enhancing bone repair in the subject in need.

In another embodiment, this invention provides a method of enhancing bone repair in a subject in need comprising the steps of: (a) obtaining a cell from said subject; (b) transfecting said cell with a vector comprising a BMP-6 ERE operably linked to a BMP-6 gene; (c) administering the engineered cell to said subject; and (d) administering to said subject an effective amount of an estrogen or estrogen agonist; thereby enhancing bone repair in said subject.

In another embodiment, this invention provides a method for maintaining or increasing bone volume, bone quality, or bone strength in a subject in need afflicted with osteoporosis caused by or accompanied by a decrease in estrogen comprising the steps of administering to said subject: (a) a BMP-6 ERE operably linked to a BMP-6 gene; and (b) an effective amount of an estrogen or estrogen agonist, thereby maintaining or increasing bone volume, bone quality, or bone strength in the subject in need.

In another embodiment, this invention provides a method for maintaining or increasing bone volume, bone quality, or bone strength in a subject in need afflicted with osteoporosis caused by or accompanied by a decrease in estrogen comprising the steps of administering to said subject: (a) an effective amount of a cell expressing an estrogen receptor, or a functional fragment thereof, further comprising a BMP-6 ERE operably linked to a BMP-6 gene; and (b) an effective amount of an estrogen or estrogen agonist, thereby maintaining or increasing bone volume, bone quality, or bone strength in the subject in need.

In another embodiment, this invention provides a method for maintaining or increasing bone volume, bone quality, or bone strength in a subject in need afflicted with osteoporosis caused by or accompanied by a decrease in estrogen comprising the steps of: (a) obtaining a cell from said subject; (b) transfecting said cell with a vector comprising a BMP-6 ERE operably linked to a BMP-6 gene; (c) administering the engineered cell to said subject; and (d) administering to said subject an effective amount of an estrogen or estrogen agonist; thereby maintaining or increasing bone volume, bone quality, or bone strength in said subject.

In another embodiment, this invention provides a method for the production of transplantable bone matrix, the method comprising the steps of: (a) obtaining a cell; (b) transfecting the cell with a vector comprising a BMP-6 ERE operably linked to a BMP-6 gene; and (c) culturing said cell with cell-associated matrix for a time effective for allowing formation of a transplantable bone matrix; thereby producing transplantable bone matrix.

In another embodiment, this invention provides a method of stimulating osteoblast differentiation comprising the steps of administering: (a) a vector comprising a BMP-6 ERE operably linked to a BMP-6 gene; and (b) an effective amount of an estrogen or estrogen agonist; thereby stimulating osteoblast differentiation.

In another embodiment, this invention provides a method of treating a bone disease in a subject in need, comprising the steps of administering to said subject: (a) a vector comprising a BMP-6 ERE operably linked to a BMP-6 gene; and (b) an effective amount of an estrogen or estrogen agonist; thereby treating a bone disease in the subject.

In another embodiment, this invention provides a method of treating a bone disease in a subject comprising the steps of administering to said subject: (a) an effective amount of a cell comprising a BMP-6 ERE operably linked to a BMP-6 gene; and (b) an effective amount of an estrogen or estrogen agonist; thereby treating a bone disease in the subject.

In another embodiment, this invention provides a method for the identification of a therapeutic agent for the prevention and/or treatment of osteoporosis, comprising: (a) introducing into an estrogen receptor expressing-cell a vector comprising a BMP-6 ERE operably linked to a BMP-6 gene; which is further operably linked to a reporter gene, (b) contacting said cell with a candidate agent; and (c) monitoring expression of the protein encoded by said reporter gene, wherein induced reporter protein expression indicates that the candidate agent is a therapeutic agent.

In another embodiment, this invention provides a method for identifying a compound in a sample as an estrogenic agonist comprising: (a) providing an estrogen receptor expressing-cell a vector comprising a BMP-6 ERE operably linked to a reporter gene, (b) contacting said cell with a candidate estrogen agonist, under conditions facilitating estrogen-mediated reporter expression; and (c) measuring reporter expression, wherein increased reporter expression as compared to a control indicates the presence of an estrogen agonist.

In another embodiment, this invention provides a method for identifying a compound in a sample as a estrogen antagonist comprising: (a) providing an estrogen receptor expressing-cell a vector comprising a BMP-6 ERE operably linked to a reporter gene, (b) contacting said cell with a candidate antagonist and an amount of estrogen, such that absent of said candidate antagonist, reporter protein expression is measurably increased; and (c) measuring reporter protein expression, wherein diminished reporter expression as compared to estrogen contact alone indicates the presence of an estrogen antagonist.

According to another embodiment, the invention provides an isolated nucleic acid comprising a BMP-6 ERE. The isolated nucleic acid, in one embodiment, corresponds to, or is homologous to an hBMP-6 intron region, or a fragment thereof, which comprises an estrogen responsive element. The isolated nucleic acid, in another embodiment, comprising the BMP-6 estrogen responsive element may be upstream of a nucleic acid sequence encoding for a BMP-6 gene.

In one embodiment, the invention provides an isolated nucleic acid as set forth in SEQ ID Nos. 1 or 2. In another embodiment, the invention provides an isolated nucleic acid at least 70% homologous to SEQ ID Nos. 1 or 2.

### BRIEF DESCRIPTION OF THE DRAWINGS

FIG. 1 shows functional ER αβ in hMSCs engineered to express the receptors following transduction with adeno-ERα and adeno-ERβ. hMSCs were infected by 100 pfu/cell adeno-ERαLacZ, adeno-ERβ&LacZ or adeno-LacZ virus with 100 pfu/cell Adeno-ERE-tk-luciferase. 10⁻⁸ M E2 or ethanol vehicle were added 24 hours after infection, luciferase activity was assayed 24 hours after E2 treatment. The results are presented as the fold induction of RLU (Relative Light Units) of E2 treated over vehicle control, after normalized their efficiency of transfection to β-gal activity.

FIG. 2 shows human ER-α & β mRNA expression in engineered hMSCs by adeno-ERα and adeno-ERβ. hMSCs were infected by 100 pfu/cell adeno-ERα, -ERβ or LacZ virus. RNA was extracted from MSCs 48 hours after infection. A. RT-PCR for human ERα, lane 1-water (negative control), lane 2-hMSCs infected with adeno-ERα no Reverse Transcriptase (RT), lane 3-hMSCs infected with adeno-ERα with RT, lane 4-hMSCs infected with adeno-LacZ, no RT, lane 5-hMSCs infected with adeno-LacZ with RT, lane 6-human ER-α expression plasmids (positive control). B. RT-PCR for human ER-β, lane 1-water (negative control), lane 2-hMSCs infected with ER-β no RT, lane 3-hMSCs with adeno-ERβ with RT, lane 4-hMSCs infected with adeno-LacZ, no RT, lane 5-hMSCs infected with adeno-LacZ with RT, lane 6-human ER-β expression plasmids (positive control).

FIG. 3 shows Real Time RT-PCR for human ER-α mRNA expression in hMSCs infected by 100 pfu/cell Adeno-ERα or Adeno-LacZ. RNA was extracted from MSCs 48 hours after infection. Human ERα mRNA level was increased from 68 copies in adeno-lacZ infected hMSCs to 1.6×10⁵ copies per μg of total RNA in adeno-ERα infected hMSCs (A&D). The internal control RPL19 indicted that the same amount of RNA was used in Real Time RT-PCR (B). The human ERα copy number (D) was obtained by using pSG5-hERα plasmids (10² to 10⁸ copies) for standard curve (C). The Real Time RT-PCR was performed 3 times in independent experiments.

FIG. 4 shows Real Time RT-PCR for human ER-β mRNA expression in hMSCs infected by 100 pfu/cell Adeno-ERβ or Adeno-LacZ. RNA was extracted from MSCs 48 hours after infection. Human ERβ mRNA level was increased from 30 copies in adeno-lacZ infected hMSCs to 8.4×10⁵ copies per pg of total RNA in adeno-ERβ infected hMSCs (A&D). The internal control RPL19 indicted that the same amount of RNA was used in Real Time RT-PCR (B). The human ERβ copy number (D) was obtained by using pSG5-hERβ plasmids (10² to 10⁸ copies) for standard curve (C). The Real Time RT-PCR was performed 3 times in independent experiments.

FIG. 5 shows upregulation by E2 of hBMP-6 mRNA expression in engineered hMSCs by adeno-ERα or adeno-ERβ as showed by RT-PCR. H MSCs were infected with 100 pfu/cell adeno-LacZ, adeno-ER-α or adeno-ER-β. 10⁻⁷ M E2 or ethanol vehicle was added into the cultures 24 hours after infection, RNA was extracted from hMSCs 24 hours after E2 treatment in vitro.

FIG. 6 shows upregulation by E2 of hBMP-6 mRNA expression in engineered hMSCs by adeno-ERα or adeno-ERβ, as showed by Real Time RT-PCR (FIGS. 6 C and D, respectively). H MSCs were infected with 100 pfu/cell adeno-LacZ, adeno-ER-α or adeno-ER-β. 10⁻⁷ M E2 or ethanol vehicle was added into the cultures 24 hours after infection and RNA was extracted from hMSCs 24 hours after E2 treatment in vitro. After 24 hours treatment, 10⁻⁷ M 17β estradiol stimulated hBMP-6 mRNA expression 3.2-fold increase in hMSCs infected Adeno-ERα (FIG. 6C, bar graph, right panel), and 2.0-fold increase in hMSCs infected with Adeno-ERβ (FIG. 6D, bar graph, right panel), but not in hMSCs infected with Adeno-LacZ (FIG. 6B, bar graph, right panel) as shown by Real Time RT-PCR. The internal control RPL19 indicated that the same amount of RNA was used in Real Time RT-PCR (lower left panel of FIGS. 6B, C, and D, respectively). The hBMP-6 copy number (bar graph, right panel of FIGS. 6B, C, and D, respectively) was obtained by using pGEM-T-hBMP-6 plasmids (10² to 10⁸ copies) for standard curve (FIG. 6A, top panel). The Real Time RT-PCR was performed 3 times in independent experiments.

FIG. 7 shows the effects of E2 in hBMP-6 promoter activity via ERα and ERβ in C3H10T1/2 cells 5 μg hBMP-6 promoter-Luc plasmids (hBMP-6 promoter hooked to luciferase in pGL3 vector), ERE-tk-Luciferase plasmids or pGL3 plasmids were transiently co-transfected into C3H10T1/2 cells with 2 μg ERα or ERβ expression vector, cells were treated with 10⁻⁸ M E2 for 24 hours, luciferase activity was assayed by luminometer. The results were showed as the fold induction of E2 treated over vehicle control. Error bars show standard error among five experiments, each done in triplicate.

FIG. 8 shows posttranscriptional mechanism: Actinomycin D does not block the up-regulation of hBMP-6 mRNA by E2 in engineered hMSCs by adeno-ERα and adeno-ERβ.

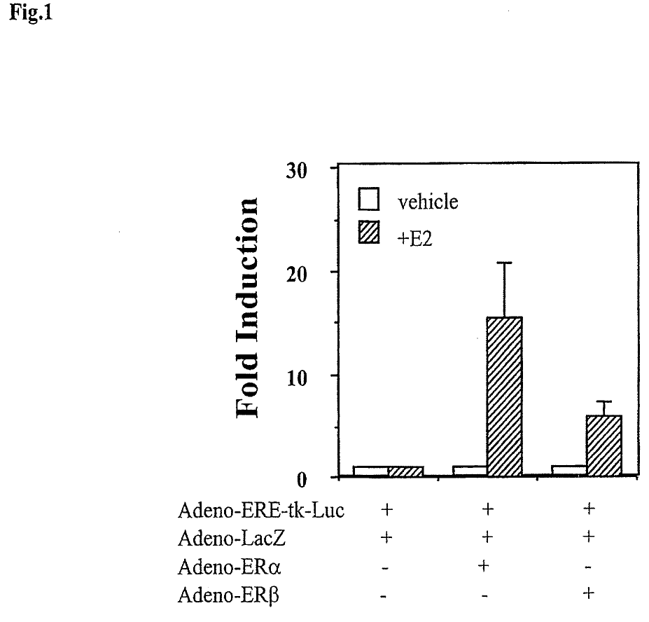

### DETAILED DESCRIPTION OF THE INVENTION

This invention provides novel nucleic acids, vectors, compositions and methods for regulating BMP-6 expression in mesenchymal stem cell populations, and therapeutic applications arising from their utilization. In one embodiment, the invention is directed to an isolated nucleic acid sequence, which comprises a BMP-6 estrogen responsive element (ERE). Estrogen belongs to the sex steroid hormone family, and is primarily responsible for maintenance of skeletal homeostasis, as reflected in the widespread and beneficial use of estrogen replacement therapy to overcome postmenopausal osteoporosis in women. Estrogen exists in multiple forms, and in one embodiment of the invention, may be considered to include any natural form of estrogen, such as, for example, 17β estradiol, 2-hydroxy estradiol, estrone, 2 hydroxy estrone, 16α hydroxy estrone, estriol, ethinyl estradiol, estradiol cypionate, estradiol valerate or estropipate, each of which is to be considered as a separate embodiment of the invention.

Estrogens, their analogs and derivatives exert similar biological effects. In one embodiment, an estrogen derivative of this invention comprises a chemical substitution of estrogen that allows for the inclusion of additional functional groups to the estrogen molecule, yet preserves a basic common functional chemical species between the derivative and estrogen. In another embodiment, estrogen mimics and estrogen analogs comprise modifications of a natural estrogen rendering a compound that is chemically distinct from estrogen yet functionally equivalent, or somewhat functionally equivalent to the natural estrogen.

In another embodiment, estrogen is any estrogen, including estrogen derivatives or estrogen analogs. The estrogen is, in another embodiment, an environmental estrogen, or includes chemical substitutions producing a compound resulting in essentially identical estrogen-mediated effects. In another embodiment, estrogens are estrogen agonists, which potentiate or equal estrogen function.

Some examples of estrogen mimics include diethylstilbestrol, 4-hydroxytamoxifen, the raloxifene analog LY353381, 2,2-bis (p-hydroxyphenyl)-1,1,1-trichloroethane (HPTE), bisphenol A, and genistein (Klotz D. M. et al, Endocrinology (2000) 141: 3430-9), and represent additional embodiments of the invention.

Estrogen mediates its effects, via binding cognate receptors followed by translocation as a complex to a cell nucleus, binding to DNA and functioning as transcription factors. Receptors bind to specific DNA sequences within the gene promoter called response elements. A canonical estrogen response element (ERE) has been defined according to the following polynucleotide string: 5′-AGGTCAnnnTGACCT-3′ (SEQ ID NO:3). The ERE is located in promoter regions of a gene in cells which will exhibit estrogen responsiveness, following estrogen binding to its cognate receptor, and translocating to the cell nucleus, whereupon stimulation of gene expression may commence.

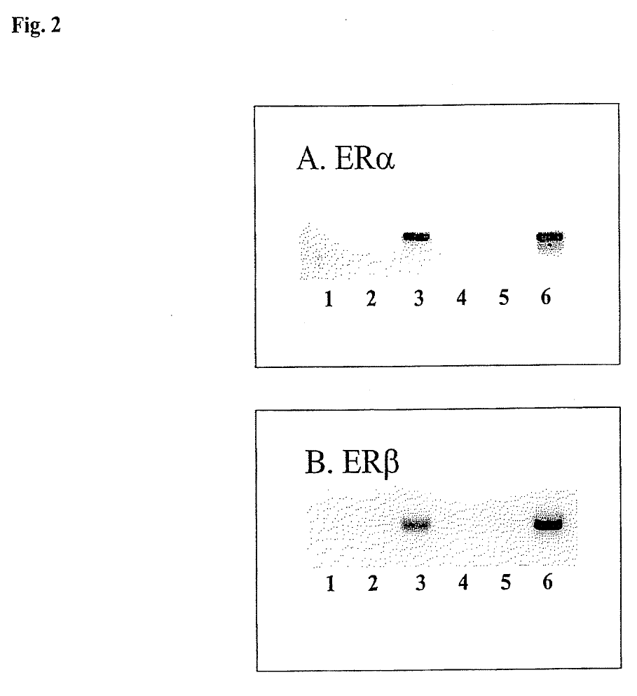

Estrogen receptor expression in hMSCs was demonstrated herein via hMSC transduction with adenoviral vectors encoding for ERα (adeno-CMV-ERα and ERβadeno-CMV-ERβ. Example 2 demonstrates effective transduction with either Adeno-ERα or Adeno-ERβ construct, and further demonstrates estrogen responsiveness as a function of adenoviral transduction (FIGS. 2-4). These results indicated that, contrary to prior suggestion, mesenchymal stem cells lack ER expression, hence estrogen responsiveness.

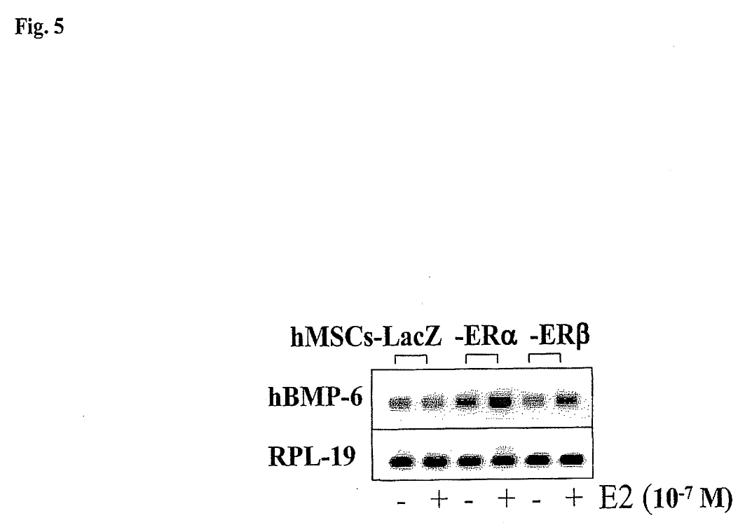

A definitive role for estrogen responsiveness in influencing BMP-6 regulation, is demonstrated herein, in Example 3 (illustrated in FIGS. 5-7), where estrogen is shown to directly stimulate hBMP-6 mRNA levels in hMSCs engineered to express estrogen receptor-α (ERα) or -β (ERβ), as compared to controls. The data indicated that estrogen-mediated effects directly involved BMP-6, in hMSC cells.

These results indicated a direct role for estrogen-mediated regulation of BMP-6 expression, a finding further corroborated by NCBI BLAST analysis of the BMP-6 gene, which produced a match for homologous sequences to Estrogen Responsive Elements (ERE). An ERE variant was found in the hBMP-6 genomic sequence, corresponding to 5′GGGGCAgtgTGACCA3′ (SEQ. ID. No. 1), with a murine orthologue identified, corresponding to 5′GGGCCActcTGACCC3′ (SEQ ID No. 2).

In one embodiment, “homology”, “homologue” or “homologous” is at least 70% correspondence with the indicated sequence. In another embodiment, the nucleic acid sequence exhibits at least 75% correspondence with the indicated sequence. In another embodiment, the nucleic acid sequence exhibits at least 80% correspondence with the indicated sequence. In another embodiment, the nucleic acid sequence exhibits at least 85% correspondence with the indicated sequence. In another embodiment, the nucleic acid sequence exhibits at least 90% correspondence with the indicated sequence. In another embodiment, the nucleic acid sequence embodiment, the nucleic acid sequence exhibits 95%-100% correspondence with the indicated sequence.

Nucleic acid sequence homology may be determined by any number of computer algorithms available and well known to those skilled in the art, for example, the Smith-Waterman algorithm, utilized in analyzing sequence alignment protocols, as in for example, the GAP, BESTFIT, FASTA and TFASTA programs in the Wisconsin Genetics Software Package release 7.0, Genetics Computer Group, 575 Science Dr., Madison, Wis. For example, the percent homology between two nucleotide sequences may be determined using the GAP program in the GCG software package, using a NWS gap DNA CMP matrix and a gap weight of 40, 50, 60, 70, or 80 and a length weight of 1, 2, 3, 4, 5, or 6.

In another embodiment, the nucleic acid sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to BMP-6 ERE nucleic acid molecules of the invention.

Nucleic acid sequence homology may be determined by hybridization to a reference sequence under highly stringent (0.2×SSC at 65° C.), stringent (e.g. 4×SSC at 65 C or 50% formiamide and 4×SSC at 42° C.), or relaxed (4×SSC at 50° C. or 30-40% formamide and 4×SSC at 42° C.) conditions.

Mutations in the BMP-6 ERE may enhance or diminish hormone responsiveness, may function to promote uptake within cells, or may provide additional benefits for their introduction, and as such are considered as part of this invention. Mutations of the BMP-6 ERE nucleic acid sequence may comprise point mutations, substitutions, insertions or deletion mutations, or induced modifications each of which represent an additional embodiment of the invention. Nucleic Acid sequences of the present invention may comprise single mutations, or multiple mutations, including combinations of the mutations listed herein, each of which is to be considered a separate embodiment of the invention.

Exposure to estrogen, according to one aspect of the invention, results ultimately in the formation of a complex of estrogen, a cognate receptor, and an ERE. It is to be understood that under such conditions, both estrogen agonists and antagonists may be supplied that may bind to an ERE, and as such are considered within the scope of the invention herein.

It is also to be understood, that according to one embodiment of the invention, estrogen responsiveness involves engagement of an estrogen receptor, capable of participating in a series of events that culminates in BMP-6 ERE engagement.

In one embodiment, the term “estrogen receptor” refers to the currently identified α or β estrogen receptors. It is also to be envisioned, however, to include any receptor subsequently identified that in response to estrogen, the receptor participates in a series of events that culminates in BMP-6 ERE engagement. Estrogen receptors of the invention may comprise truncated or chimeric estrogen receptors, or natural estrogen receptors as described in Berry, et al., E.M.B.O. J., 9:2811-2818 (1990). Estrogen receptor modification may result in increased estrogen affinity and subsequent estrogen responsiveness.

Estrogen administration can be accomplished in vitro, for example, by direct injection into a cell, or any other means well known to one skilled in the art. Indirect administration may be accomplished in vitro, for example, by supplementing a media surrounding a given cell with an estrogen, which enables the cell to ultimately be exposed to the estrogen. Similarly, in vivo, administration may be conducted directly to a specific tissue site, via, for example, injection or catheterization, or indirectly via parenteral administration, by for example, osmotic pump delivery, aerosol exposure, and numerous methods well described in the art.

In one embodiment, “BMP-6 ERE” includes any nucleic acid sequence, incorporated within a cell, by any means, which renders expression of BMP-6 within that cell to be inducible by an estrogen, as defined herein. Estrogen responsiveness, as such, may be rendered to a given cell by its incorporation of a BMP-6 ERE of the present invention, whereby a cell expressing the ERE may show induction of BMP-6 expression following the administration of an estrogen. The BMP-6 ERE of the present invention comprises the nucleic acid sequence, vectors comprising that sequence, cells comprising that sequence, and/or compositions comprising the sequence, each of which represents a separate embodiment of this invention.

In addition to nucleic acid sequences comprising a BMP-6 ERE, the invention provides, in another embodiment, an oligonucleotide that comprises only a portion of the nucleic acid sequences of SEQ ID Nos: 1 or 2, for example a fragment which can be used as a probe or primer or a fragment encompassing a biologically active portion of the BMP-6 ERE. The nucleotide sequence allows for the generation of probes and primers designed for use in identifying and/or cloning other BMP-ERE members, as well as BMP-ERE family homologues from other species. The probe/primer typically comprises a substantially purified oligonucleotide of at least about 12 or 15, preferably about 20 or 25, more preferably about 30, 35, 40, 45, 50, 55, 60, 65, or 75 nucleotides in length, specifically hybridizing with a BMP-6 ERE nucleic acid.

The oligonucleotide of this invention, according to another embodiment, may be in either sense or antisense orientation, may comprise DNA or RNA, and/or may be single or double stranded. The invention also provides, in other embodiments, compositions and/or vectors comprising the oligonucleotides of this invention.

To generate the vectors of the present invention, the polynucleotide segments encoding BMP-6 ERE and other sequences of interest can be ligated into commercially available expression vector systems suitable for transducing/transforming eukaryotic or prokaryotic cells and for directing the expression of recombinant products within the transduced/transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter genes.

According to another embodiment, nucleic acid vectors comprising the isolated nucleic acid sequences delineated herein include a promoter for regulating expression of the isolated nucleic acid. Such promoters are known to be cis-acting sequence elements required for transcription as they serve to bind DNA dependent RNA polymerase, which transcribes sequences present downstream thereof. Should a desired vector contain an undesirable promoter, such promoter can be excised using standard methods and replaced by a nucleic acid sequence comprising the BMP-6 ERE hormone responsive element.

A vector according to the present invention preferably further includes an appropriate selectable marker. The vector may further include an origin of replication, and may be a shuttle vector, which can propagate both in prokaryotic, and in eukaryotic cells, or the vector may be constructed to facilitate its integration within the genome of an organism of choice. The vector according to this aspect of the present invention can be, for example, a plasmid, a bacmid, a phagemid, a cosmid, a phage, a virus or an artificial chromosome.

In one embodiment, BMP-6 ERE incorporation in mammalian cells can be accomplished using various viral vectors. The viral vector is engineered to contain nucleic acid, e.g., a cDNA, of the desired nucleic acid sequence. Transfection of cells with a viral vector has the advantage that a large proportion of cells receive the nucleic acid, which can obviate the need for selection of cells which have received the nucleic acid. Additionally, molecules encoded within the viral vector, e.g., a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for review see Miller, A. D. (1990) Blood 76:271). A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions which can be used to infect a target cell through the use of a helper virus by standard techniques.

Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danosand Mulligan (1988) Proc. Natl. Acad. Sci. USA 85:6460-6464; Wilson et al. (1988) Proc. Natl. Acad. Sci USA 85:3014-3018; Armentano et al., (1990) Proc. Natl. Acad. Sci. USA 87: 6141-6145; Huber et al. (1991) Proc. Natl. Acad. Sci. USA 88:8039-8043; Feri et al. (1991) Proc. Natl. Acad. Sci. USA 88:8377-8381; Chowdhury et al (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Non-viral vectors may also be used to transform desired cells with recombinant nucleic acids and are additional preferred embodiments of the present invention. These sequences may also be engineered to include the necessary regulatory elements within the non-viral vector. Examples of such non-viral vectors include, and not by way of limitation: Plasmids such as CDM8 (Seed, B. (1987) Nature 329:840) and pMT2PC (Kaufman, et al. (1987) EMBO J. 6:187-195). Additional suitable commercially available mammalian expression vectors include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives. Linear DNA expression cassettes (LDNA) may be employed as well (Zhi-Ying Chen et al ASGT 2001 Abst.).

Nucleotide sequences are typically operably linked to, i.e., positioned, to ensure the functioning of an expression control sequence. These expression constructs are typically replicable in the cells either as episomes or as an integral part of the cell's chromosomal DNA, and may contain appropriate origins of replication for the respective prokaryotic strain employed for expression. Commonly, expression constructs contain selection markers, such as for example, tetracycline resistance, ampicillin resistance, kananiycin resistance or chlormaphenicol resistance, facilitating detection and/or selection of those bacterial cells transformed with the desired nucleic acid sequences (see, e.g., U.S. Pat. No. 4,704,362). These markers, however, are not exclusionary, and numerous others may be employed, as known to those skilled in the art. Indeed, in a preferred embodiment of the present invention expression constructs contain both positive and negative selection markers.

The nucleic acid vector may be introduced into desired cells by direct DNA uptake techniques, and virus, plasmid, linear DNA or liposome mediated transduction, or transfection, magnetoporation methods employing calcium-phosphate mediated and DEAE-dextran mediated methods of introduction, electroporation, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals).

In another embodiment, cells are transduced or transformed by a vector comprising a reporter gene, wherein the reporter gene is operatively linked to a BMP-6-ERE, or a fragment thereof. The BMP-6 ERE, as described above is inducible by an estrogen, producing increased levels of the reporter gene product in the presence of an estrogen.

In another embodiment, the vector comprising a BMP-6 ERE is operably linked to a reporter gene may be utilized to transiently or stably transfect appropriate host cells via means well known in the art, such as those described hereinabove.

Nucleic acids are considered operably linked when they are functionally related to each other. For example, a promoter is operably linked to a coding sequence if it controls the transcription of the sequence; a ribosome-binding site is operably linked to a coding sequence if it is positioned so as to permit translation. Generally, operably linked means contiguous.

In another embodiment, the isolated nucleic acid comprising a BMP-6 ERE is operatively linked to a second nucleic acid.

By utilization of the term “a second nucleic acid”, it is to be understood that any nucleic acid sequence, may be operatively linked to a BMP-6 ERE. In one embodiment, the second nucleic acid may comprise a nucleic acid, which is associated with clinical estrogen insufficiency or with a lack of estrogen responsiveness in a subject. In another embodiment, the second nucleic acid may comprise those encoding osteogenic factors or genes associated with other actions of estrogen such as those associated with cognitive functions, neuroprotection, enhancement of nerve regeneration and stimulation of neurite growth. In another embodiment the second nucleic acid may a gene associated with cancer, angiogenesis, stroke or cardiovascular disease.

In another embodiment, the second nucleic acid may correspond to a nucleic acid, which encodes osteogenic factors such as OP-1, OP-2, BMP-5, BMP-6, BMP-2, BMP-3, BMP-4, BMP-9, DPP, Vg-1, 60A or Vgr-1.

In one embodiment, following exposure to estrogen, gene product expression of a gene operatively linked to a BMP-6 ERE is increased by at least 1.5 fold. In another embodiment, BMP-6 expression is increased by 1.5 fold to 30 fold, as a function of being operatively linked to a BMP-6 ERE, following exposure to estrogen.

Large-scale production of a product of a gene operatively linked to a BMP-6 ERE can be accomplished, by methods herein described, in one embodiment, utilizing for example, mammalian cells cultured in vitro engineered to express a BMP-6 ERE operatively lined to a second nucleic acid (e.g., encoding a protein of interest). In another embodiment, an insect cell/baculovirus expression system may similarly be used, by methods well described in the art.

To produce and isolate a gene product of interest, a host cell expressing a BMP-6 ERE operatively linked to a second nucleic acid encoding a gene product of interest, is first grown in a culture medium in the absence of an estrogen. Under these conditions, expression of the second nucleic acid is repressed. Next, the concentration of an estrogen in the culture medium is increased to stimulate transcription of the second nucleic acid. The gene product can then be isolated from harvested cells or from the culture medium by standard techniques.

The invention also provides, in another embodiment, for large-scale production of a protein of interest in animals, such as in transgenic farm animals. Advances in transgenic technology have made it possible to produce transgenic livestock, such as cattle, goats, pigs and sheep (reviewed in Wall, R. J. et al. (1992) J. Cell. Biochem. 49:113-120; and Clark, A. J. et al. (1987) Trends in Biotechnology 5:20-24). Accordingly, transgenic livestock carrying in their genome the components described hereinabove can be constructed.

A transgenic animal can be created, for example, by introducing a nucleic acid encoding a protein of interest operatively linked to a BMP-6 ERE of the invention, into the male pronuclei of a fertilized oocyte, e.g., by microinjection, and allowing the oocyte to develop in a pseudopregnant female foster animal. Intronic sequences and polyadenylation signals can also be included in the transgene to increase the efficiency of expression of the transgene. Methods for generating transgenic animals, particularly animals such as mice, have become conventional in the art and are described, for example, in U.S. Pat. Nos. 4,736,866 and 4,870,009 and Hogan, B. et al., (1986) A Laboratory Manual, Cold Spring Harbor, N.Y., Cold Spring Harbor Laboratory. A transgenic founder animal can be used to breed additional animals carrying the transgene. A transgenic animal carrying one transgene can further be bred to another transgenic animal carrying a second transgenes to create a so-called “double transgenic” animal carrying two transgenes.

The invention provides, in another embodiments, a cell comprising a BMP-6 ERE that corresponds to or is homologous to SEQ ID Nos. 1 or 2.

In one embodiment, the cell of the invention may be an osteoblast, a mesenchymal stem cell, a progenitor cell or any cell, which is capable of differentiating into an osteoblast. Such differentiation may be effected by provision of any number of growth factors, well described in the art, and as such is considered as part of this invention.

The invention further provides, in another embodiment, a method of regulating expression of BMP-6 in a subject comprising the steps of administering a vector comprising a BMP-6 ERE operably linked to a BMP-6 ERE gene and an effective amount of an estrogen to the subject, thereby regulating expression of BMP-6 in the subject.

According to this aspect of the invention, administration of an estrogen culminates in its binding to the ERE upstream of the BMP-6 gene, and effectively enhances BMP-6 expression, with estrogen compounds exhibiting agonist or natural estrogen activity. With the use of estrogen antagonists, BMP-6 expression is inhibited. The cells according to this aspect of the invention express a functional receptor facilitating estrogen internalization, culminating in the described effects of BMP-6 gene regulation.

In another embodiment, this invention provides a method of regulating expression of BMP-6 in a subject comprising the steps of administering a vector comprising a nucleic acid sequence encoding for an estrogen receptor, or a functional fragment thereof, to at least one cell comprising a BMP-6 ERE operatively lined to a BMP-6 gene and an effective amount of an estrogen to the subject.

According to this aspect of the invention, cells not endogenously expressing estrogen receptors may be engineered to express the receptors, in order that estrogen administration culminates in its binding to the ERE upstream of the BMP-6 gene, regulating BMP-6 expression, as described.

BMP-6 expression can, in one embodiment, be regulated in cells through an estrogen-dependent mechanism, in cells not endogenously responsive to an estrogen. For example, as demonstrated herein in Example 3, human mesenchymal stem cells can be engineered to express high levels of BMP-6 via introduction of estrogen receptors, in cells expressing a BMP-6 ERE containing vector. Only following introduction of the estrogen receptor was there demonstrable increased BMP-6 gene expression, as cells comprising the BMP-6 ERE, without further manipulation to express an estrogen receptor failed to provide detectable BMP-6 gene expression.

In another embodiment, this invention provides a method of regulating expression of BMP-6 in a subject comprising the steps of administering an effective amount of a cell expressing an estrogen receptor or a functional fragment thereof, further comprising a BMP-6 ERE operatively linked to a BMP-6 gene and an effective amount of an estrogen to the subject, thereby regulating expression of BMP-6 in the subject.

According to this aspect of the invention, in one embodiment, cells endogenously expressing an estrogen receptor can be engineered to express BMP-6 through an estrogen-mediated pathway.

Any cell whereby BMP-6 expression is desirable may be utilized, whether in one embodiment, endogenously expressing an estrogen receptor or a functional fragment thereof, or, in another embodiment, whether engineered to express same. Some examples of cells thus utilized may include, for example, stem cells, in particular mesenchymal stem cells, progenitor cells, osteoblasts, or any cell that may be differentiated into an osteoblast, each of which represents a separate embodiment of the invention.

The invention is widely applicable to a variety of situations where it is desirable to be able to turn gene expression “on” and/or “off”, or regulate the level of gene expression, in a rapid, efficient and controlled manner without causing pleiotropic effects or cytotoxicity. The invention may be useful for gene therapy purposes in humans, in treatments for either genetic or acquired diseases. The general approach of gene therapy involves the introduction of one or more nucleic acid molecules into cells such that one or more gene products encoded by the introduced genetic material are produced in the cells to restore or enhance a functional activity. For reviews on gene therapy approaches see Anderson, W. F. (1992) Science 256:808-813; Miller, A. D. (1992) Nature 357:455-460; Friedmann, T. (1989) Science 244:1275-1281; and Coumoyer, D., et al. (1990) Curr. Opin. Biotech. 1:196-208. However, current gene therapy vectors typically utilize constitutive regulatory elements which are responsive to endogenous transcriptions factors. These vector systems do not allow for the ability to modulate the level of gene expression in a subject. In contrast, the regulatory system of the invention provides this ability.

In one embodiment, the cell or vector of the invention may comprise a promoter, which is tissue or organ specific (for example, brain, heart or blood vessel) so as to enable the expression of desired genes in specific organs or tissue. In another embodiment, cells or vectors may be directed specifically to a given tissue or organ via delivery methods well known in the art, some as described hereinabove.

In another embodiment, the invention provides a method of down modulating or inhibiting an estrogen-mediated response, or an oversensitive estrogen-mediated response of certain genes. According to this aspect of the invention, ERE affinity of the vectors of this invention can be utilized as a decoy for estrogen receptor binding, where the ERE is expressed, but no downstream gene is affected. ERE expression serves as a bait for estrogen receptors that following binding to an estrogen would produce downstream effects that are undesired. Binding of such complexed ERs to the decoy competes their binding to endogenous EREs preventing their downstream effects on host cell genomes.

In another embodiment, the invention is related to the field of gynecology and fertility. The estrogen responsive element may be used to regulate expression of genes such as hormones, for example without being limited, of LH or FSH.

In another embodiment, this invention provides a method of increasing a responsiveness of a cell to an estrogen comprising the step of contacting a cell with a vector comprising a BMP-6 ERE, thereby increasing the responsiveness of the cell to an estrogen.

Increasing estrogen responsiveness in a cell may be accomplished in one embodiment, in a cell within a subject, or in another embodiment, in a cell from a subject. In another embodiment any cell including, but not limited to, fungal cells, plant cells, insect cells, e.g. Schneider and sF9 cells, or mammalian cells, such as HeLa cells (human), NIH3T3 (murine), RK13 (rabbit) cells may be utilized. Embryonic stem cells or embryonic stem cell lines, e.g., D3 and J1, including pluripotent, or partially differentiated stem cells, such as hematopoietic stem cells, myoblasts, lymphoblasts, etc. may be utilized. In other embodiments, progenitor cells or differentiated cells may also comprise a cell of this invention, wherein it is desired to increase cellular estrogen responsiveness, including, for example, hepatocytes, lymphocytes, airway epithelium or skin epithelium or any recombinant eukaryotic cell.

In another embodiment, a cell modified to enhance estrogen responsiveness may be implanted in a subject through any of the means well described in the art, including catheterization, direct injection, and others.

In another embodiment, this invention provides a method of enhancing bone repair in a body of a subject in need comprising the steps of administering a vector comprising BMP-6 ERE operably linked to a BMP-6 gene and an effective amount of an estrogen to the subject, inducing BMP-6 expression, thereby enhancing bone repair.

The need for bone repair, in one embodiment, may be as a result of any disease state, such as osteoporosis, wherein estrogen-mediated bone remodeling is affected In another embodiment, bone repair may be effected in a host wherein estrogen-mediated bone remodeling is unaffected, such as in bone fractures that fail to heal spontaneously, yet where the proposed method provides for bone repair, without deleterious effects to the subject.

In another embodiment, this invention provides a method of enhancing bone repair in a subject in need comprising the steps of administering an effective amount of a cell comprising a BMP-6 ERE operably linked to a BMP-6 gene and an effective amount of an estrogen to the subject, inducing BMP-6 expression, thereby enhancing bone repair.

In another embodiment, bone repair may be enhanced in a subject in need by a method comprising the steps of transfecting a cell obtained from the subject with a vector comprising a BMP-6 ERE operably linked to a BMP-6 gene, administering the engineered cell to the subject; and administering an effective amount of an estrogen to the subject, inducing BMP-6 expression, thereby enhancing repair of a bone in the body of the subject in need.

In another embodiment, this invention provides methods for maintaining or increasing bone volume, bone quality, or bone strength in a subject in need, afflicted with osteoporosis caused by or accompanied by a decrease in estrogen or estrogen responsiveness.

The methods according to this aspect, in one embodiment, comprise the steps of administering a vector comprising a BMP-6 ERE operably linked to a BMP-6 gene and an effective amount of an estrogen to the subject, inducing BMP-6 expression, thereby maintaining or increasing bone volume, bone quality, or bone strength in the subject.

In another embodiment, the method for maintaining or increasing bone volume, bone quality, or bone strength comprises the steps of administering an effective amount of a cell comprising a BMP-6 ERE operably lined to a BMP-6 gene and an effective amount of an estrogen, inducing BMP-6 expression, thereby maintaining or increasing bone volume, bone quality, or bone strength in the subject.

In another embodiment, the method for maintaining or increasing bone volume, bone quality, or bone strength in a subject afflicted with osteoporosis caused by or accompanied by a decrease in estrogen or estrogen responsiveness comprises the steps of transfecting a cell obtained from the subject with a vector comprising a BMP-6 ERE operably linked to a BMP-6 gene and administering an effective amount of an estrogen to the subject, inducing BMP-6 expression, thereby maintaining or increasing bone volume, bone quality, or bone strength in a subject in need.

Changes in bone volume, quality or strength, and as a function of bone repair may be measured by a number of methodologies well known to one skilled in the art, including methods directly measuring tensile strength, and methods measuring various bone markers, as described in U.S. Pat. No. 5,785,041 by Weinstein et al., U.S. Pat. No. 5,509,042 by Mazess et al., Ronis M. J. J. et al Toxicol Sci, (2001) 62: 321-329 or Suponitsky I. et al Journal of Endocrinology (1998) 156: 51-57. Bone repair may be taken as an improvement in measurements as above, as a function of the methods listed herein.

In one embodiment, this invention provides a method for the production of transplantable bone matrix, the method comprising the steps of transfecting a cell with a vector comprising a BMP-6 ERE operably linked to a BMP-6 gene and culturing the cell with cell-associated matrix for a time effective for allowing formation of a transplantable bone matrix.

In another embodiment, this invention provides a method of stimulating osteoblast differentiation comprising the steps of administering a vector comprising a BMP-6 ERE and an effective amount of an estrogen to an undifferentiated osteoblast, inducing BMP-6 expression, thereby stimulating osteoblast differentiation.

Osteoblast differentiation may be measured via assessment of cell surface expression of osteopontin and BSP-II, which may be determined by FACS analysis, immunohistochemistry or immunofluorescence assay, alternatively differentiation may be determined via assaying cell alkaline phosphatase (ALP) activity by known histological techniques, or via assaying interleukin-6 (IL-6) and osteocalcin gene expression via RT-PCR, Northern blot analysis or RNase protection assay of via assaying protein levels via Western blot analysis, ELISA or RIA.

In another embodiment, this invention provides a method of treating a bone disease in a subject. In one embodiment, the method comprises the steps of administering a vector comprising a BMP-6 ERE operably linked to a BMP-6 gene, inducing BMP-6 expression, thereby treating a bone disease in the subject.

In another embodiment, the method of treating a bone disease comprises the steps of administering an effective amount of a cell comprising a BMP-6 ERE operatively linked to a BMP-6 gene and an effective amount of an estrogen to the subject, inducing BMP-6 expression, thereby treating bone disease in the subject.

In another embodiment, this invention provides a method for the identification of a potential therapeutic agent for the prevention and/or treatment of osteoporosis, comprising: introducing a vector comprising a BMP-6 ERE operably linked to a BMP-6 gene and a reporter gene into an estrogen receptor-expressing cell, contacting the cell with a candidate agent and monitoring reporter protein expression, wherein induced expression indicates that the candidate agent is a potential therapeutic agent.

In screening for therapeutic agents for osteoporosis, cells are provided which are transformed, in other embodiments, with any of the recombinant vectors of the invention. The cells are plated in a number of culture dishes or in multi-well culture plates in a culture medium appropriate to the cell type used and then contacted with samples suspected to contain therapeutic agents for osteoporosis. These samples can be, e.g., aqueous or water-miscible solutions in which isolated compounds have been dissolved, or individual or pooled fractions from purification steps such as chromatography or preparative electrophoresis. Negative (sample buffer only) and positive (known amounts of estrogen or estrogen agonist) controls may be assayed in parallel.

After incubation of the cells for an induction period, the level of expression of the reporter gene produced by each sample is measured by an assay appropriate for the gene used. The optimal time for making the measurement is determined by routine experimentation but will typically be in the range of about 24 to 72 hours. Therapeutic agents for osteoporosis in a sample will be identified by measuring a level of reporter gene expression that is higher than the unstimulated (buffer control) level.

In another embodiment, this invention provides a method for identifying a compound as an estrogen agonist, in a sample, the method comprising: introducing a vector comprising a BMP-6 ERE operatively linked to a reporter gene into a cell expressing an estrogen receptor, contacting the transfected cell with a sample suspected to contain an estrogen agonist, under conditions facilitating estrogen mediated reporter expression and measuring reporter expression, wherein increased reporter expression as compared to a control, indicates the presence of an estrogen agonist.

In another embodiment, this invention provides an efficient means of screening large numbers of test compounds for exhibition of desirable properties for the treatment or prevention of various cancers (e.g. breast cancer, ovarian cancer, endometrial cancer) or other diseases (e.g. endometriosis) mediated by estrogen.

In another embodiment, this invention provides a method for identifying a compound in a sample as a estrogen antagonist comprising introducing a vector comprising a BMP-6 ERE operatively linked to reporter gene into an estrogen receptor expressing cell, contacting the transfected cell with a sample suspected to contain an estrogen antagonist, such that absent of the suspected antagonist, reporter protein expression is measurably increased, and measuring reporter expression, wherein diminished reporter expression as compared to estrogen contact alone indicates the presence of an estrogen antagonist.

In another embodiment, the invention provides methods of screening for compounds blocking estrogen-mediated effects, dictated at the level of an estrogen responsive element. When testing any compound for estrogenic activity, whether stimulatory or inhibitory, or exhibiting potential osteoporosis therapeutic activity, in other embodiments of the invention the cells utilized may include those expressing high levels of an estrogen receptor, such as the following non-limiting examples: MCF-7 cells (ATCC No. HTB 22), MDA453 cells (ATCC No. HTB 131), ZR-75-1 cells (ATCC No. CRL 1500) ERC1 cells (described in Kushner et al., Mol. Endocrinol., 4:1465-1473 (1990) ERC2 or ERC3 cells (as described by Webb, et al. Mol. Endocrinol., 6:157-167 (1993)).

Cells expressing mutant estrogen receptors with decreased sensitivity for estrogenic compounds comprise additional embodiments of the invention, and may be similarly employed, as may cells expressing a wild type receptor (e.g., MCF7 cells). In other embodiments, cells may over-express mutant estrogen receptors.

In another embodiment, cells are transfected with a reporter gene under the control of a response element other than the BMP-ERE (such as, for example, AP1) regulating reporter expression. Two different reporter genes may be utilized according to this aspect of the invention, wherein one reporter indicates transcription induced by an estrogen response system of the invention, while another indicates transcription induced by an indirect estrogen response.

The following examples further illustrate some aspects of the invention herein described, including demonstration of estrogen-mediated regulation of BMP-6 expression, via its interaction with a BMP-6 ERE. These examples combined with the specification hereinabove, are intended for exemplification purposes, and are not to be construed as a means of limiting the present invention.

### EXAMPLES

### Materials and Methods

### Chemical Reagents:

All materials were purchased from Sigma Chemical Co. (St. Louis, Mo.) unless otherwise indicated. DMEM, Penicillin-streptomycin, L-glutamine were purchased from Biological Industries (Beit Haemek, Israel).

### Cell Lines:

Mouse C3H10T1/2 cells were cultivated in DMEM (Sigma and Biological Industries) with 10% FBS, 100 units/ml penicillin, 100 μg/ml streptomycin, and 2 mM glutamine.

### Adenoviral Vectors:

The Adenoviral constructs expressing an estrogen receptor, under control of the CMV promoter, Ad5-CMV-ERα, AD5-CMV-ERβ (long form) and the adenoviral vector containing an estrogen response element, expressing thymidine kinase and luciferase, AD5-ERE-tk-Luciferase virus were kind gifts of the Women Health Institute, Wyeth, U.S.A.

hBMP-6 Promoter-Luc Vector Construction:

For construction of the hBMB-6-Luc constructs: the promoter fragment of hBMP-6 (−1040 to +82) was subcloned into the pGL3-basic vector as previously described (Yamada et al 1998). HBMP-6 PCR fragments were obtained via forward primer: 5′-AAGCCTCGAGTCCGCTAAACGCCTAC (SEQ ID NO:4), (containing an Xho I site) and reverse primer: 5′-TCAGATCTTATCTCTCATGGTCGTC (SEQ ID NO:5), (containing a Bgl II site). PCR utilizing the Roche FastStart Taq polymerase was conducted in an MJ minicycler (MJ Research, USA) under 1 cycle incubation at 95° C. for 5 minutes, and 35 cycles at 95° C. 1 minute per cycle, then one cycle each at 60° C. for 1 minute and 72° C., 2 minutes. PCR products were subcloned into pGEM-T Easy vector first for sequence assays, then digested with Xho I and Bgl II and subsequently ligated into the pGL3 basic vector (Promega, USA).

hMSC Culture and Adenoviral Infection:

Human mesenchymal stem cells (hMSCs) were isolated from explants of human bone marrow (approved by the Helsinki Committee Board of the Hadassah Medical Center, Jerusalem, Israel) and expanded in vitro. Isolation of hMSCs was performed as previously described (Pittenger et al., 1999). Briefly, 10 ml marrow aspirates were collected into a tube with 6000 Units heparin, washed with phosphate buffered saline (PBS), and recovered cells were collected by centrifugation at 900×g. Collected cells were then loaded onto Percoll solution (density 1.073 g/ml). Cell separation was accomplished by centrifugation at 1100×g (30 minutes at 20° C.). Mononuclear cells were collected, washed twice with PBS, cultured and sub-cultured in Dulbecco's minimal essential medium (DMEM) (low glucose) supplemented with 10% fetal calf serum (FCS) in 12 wells plate (NUNC, USA).

hMSCs were infected in vitro at 80% confluence with Ad5-CMV-ERα, Ad5-CMV-ERβ and Ad5-CMV-LacZ at 100 pfu/cell, for 1 hour in serum-free DMEM after which DMEM supplemented with 10% FCS was added, as previously described (Bodine et al., 1997 and 1999). Efficiency of infection was estimated via β-gal activity determination, utilizing the Galacto-Light Chemiluminescent Reporter Assay System Kit (Tropix of PE Biosystems, USA). Results are represented as fold-induction of RLU (Relative Light Units) of E2 treated over control, where RLU values are normalized to reflect transfections efficiency. Five experiments, each in triplicate were conducted, standard error is represented.

### Transfection and Luciferase Assays

hMSC transfection with luciferase reporter plasmids was performed as previously described (An et al, 1999). 5 μg luciferase reporter plasmids were transiently co-transfected into hMSCs cells with 2 μg ERα or ERβ expression vectors via electroporation. Cells were then treated with 17β-estradiol (E2) (1^{-7 or -8} M) or an ethanol control for 24 hours, and luciferase activity was assayed by luminometer (Turner Designs TD-20/20, CA).

Transfection efficiency was determined in parallel, in hMSCs transfected with 0.5 μg of pNGVL1-nt-BetaGal plasmid (Constructed by National Gene Vector Laboratory at the University of Michigan, Ann Arbor, USA, which was supported by NIH grant #U42RR11149), where β-galactosidase activity was assayed via Galacto-Light Chemiluminescent Reporter Assay System Kit, and luminescence recorded as above (Tropix of PE Biosystems, USA).

C3H10T1/2 cells were transfected with 5 μg of the construct hBMP-6 promoter-Luc, which is a pGL3 backbone plasmid containing luciferase under the control of the hBMP-6 promoter, 2 μg ERE-tk-Luc plasmids or pGL3 basic plasmids (promega), by methods previously described (An et al., 1999).

### RNA Isolation, RT-PCR and Real Time PCR:

RNA was isolated utilizing the TRIzol Reagent (Life Technologies, USA), according to manufacturer's protocol. RT-PCR was performed as described previously (Zhou et al., 2001). Forward and reverse primers for human ERα were: 5′-AAGGAGACTCGCTACTGTGCAG-3′ (SEQ ID NO:6) and 5′-ATCAGGATCTCTAGCCAGGCAC-3′ (SEQ ID NO:7), respectively, and were designed according to the human ERα sequence (Green et al., 1986). Forward and reverse primers for human ERβ were: 5′-TTCCCAGCAATGTCACTAACT-3′ (SEQ ID NO:8), and 5′-TCTCTGTCTCCGCACAAGG-3′ (SEQ ID NO:9), respectively, designed according to human ERβ sequence (Moore et al., 1998). Primers for the hBMP-6 (458 bp) (Rickard et al., 1998), and internal control RPL19 (190 bp) (Orly et al., 1994) sequences were as described previously.

PCR conditions used for hERα and hERβ amplification were as follows: 30 cycles at 94° C. for 1 minute, 60° C. for 1 minute and 72° C. for 2 minute. Conditions used for hBMP-6 reverse transcription and PCR amplification were: incubation with RT, followed by 30 cycles of 94° C. for 1 minute, 55° C. for 1 minute and 72° C. for 2 minute in an MJ MiniCycler (MJ Research, USA). RT-PCR products of hBMP-6 were cloned into pGEM-T Easy vector (A1360, Promega), which following sequencing confirmed that the RT-PCR product was the hBMP-6 sequence.

Real Time PCR was performed by using Roche LightCycler according to manufacturer's protocol (Roche Molecular biochemicals, USA). After reverse transcription reaction by using 2 ug total RNA, Real time PCR was carried out in 20 μl final volume by using LightCycler-FastStart DNA Master SYBR Green I kit (Roche). The reaction mix contained 1×LightCycler-FastStart Master SYBR Green I, 0.5 μM of each primer, 4 mM MgCl2, and 2 μl cDNA from RT reaction. The conditions of Real Time PCR as following: 95° C. 10 minutes for one cycle to activate the modified FastStart Taq DNA polymerase, followed by 45 cycles of 95° C. for 15 seconds, 60-55° C. touchdown at step size 0.5° C. for 10 seconds for hBMP-6, 65-60° C. touchdown at step size 0.5° C. for 10 seconds for hERα, hERβ and RPL19, and then 72° C. for 25 seconds, following which fluorescence was determined at 82° C. for 5 seconds. To quantitative the copy number of hBMP-6, hERα, or hERβ gene in hMSCs, pGEM-T-hBMP-6, pSG5-hERα or pSG5-hERβ (An et al., 1999) plasmids (10² to 10⁸ copies) were used to generate a standard curve.

### Statistical Analysis:

All experiments were performed between three and five times. Data are presented as mean values±the standard error of the mean. The RT-PCR and Real Time RT-PCR were performed 3 times in independent experiments by using total RNA isolated from 3-6 animals at each timepoint assayed. Quantitative data were analyzed by using either the non-parameteric Mann-Whitney test or ANOVA.

### Example 1

### hMSCs can be Engineered to Exhibit Estradiol Responsiveness

In order to determine the mechanism of E2-mediated stimulation of hBMP-6 expression, a comprehensive analysis was conducted on the known hBMP-6 gene sequence to establish whether a sequence consistent with an Estrogen Responsive Element (ERE) could be identified. BLAST analysis provided a match for an ERE in the genomic sequence of the hBMP-6 gene, by search query with the following sequence: 5′-nGGnCAnnnTGACCn-3′ (SEQ ID NO:10), where “n” indicates a variable base within the classical ERE sequence.

hMSCs containing the ERE-tk-luciferase construct and the adeno-lacZ construct failed to produce luciferase activity, following E2 treatment, which indicted that no functional estrogen receptors (Ers) were expressed on hMSCs, irrespective of donor source (14 year old female, 84 year old female, 19 year old male, 79 year old male and 30 year old male).

E2 stimulated a 16-fold increase in luciferase activity in hMSCs infected with adeno-ERE-tk-luc following co-infection with adeno-ERα and adeno-LacZ and a 6-fold increase following similar co-infection with adeno-ERβ and adeno-LacZ. These results indicated that hMSCs may acquire responsiveness to estrogen following efficient transduction with adenoviruses encoding for human ERα and ERβ.

### Example 2

### Efficient Transduction by Adeno-ERα/β of Non-Endogenous Expressing hMSCs

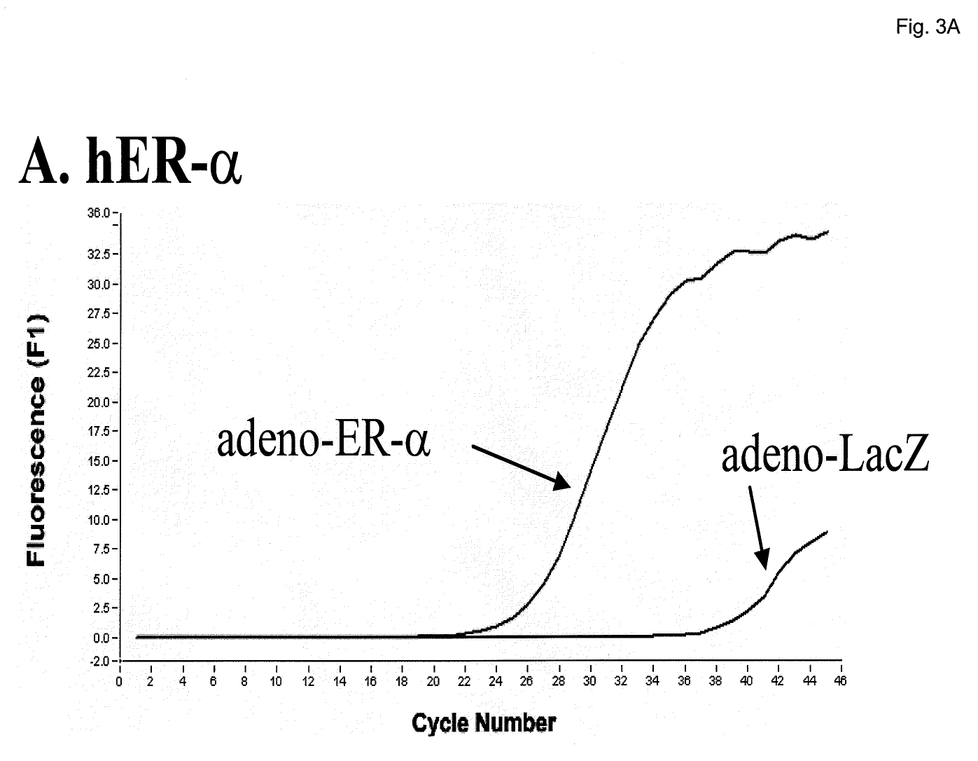

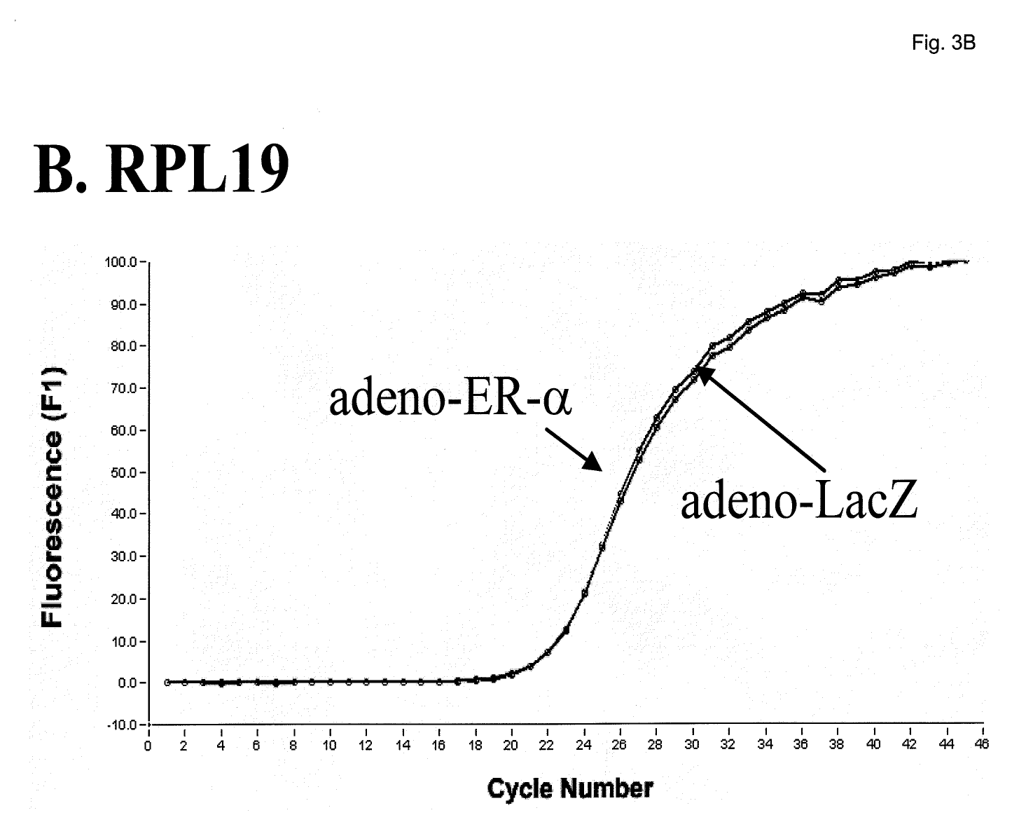

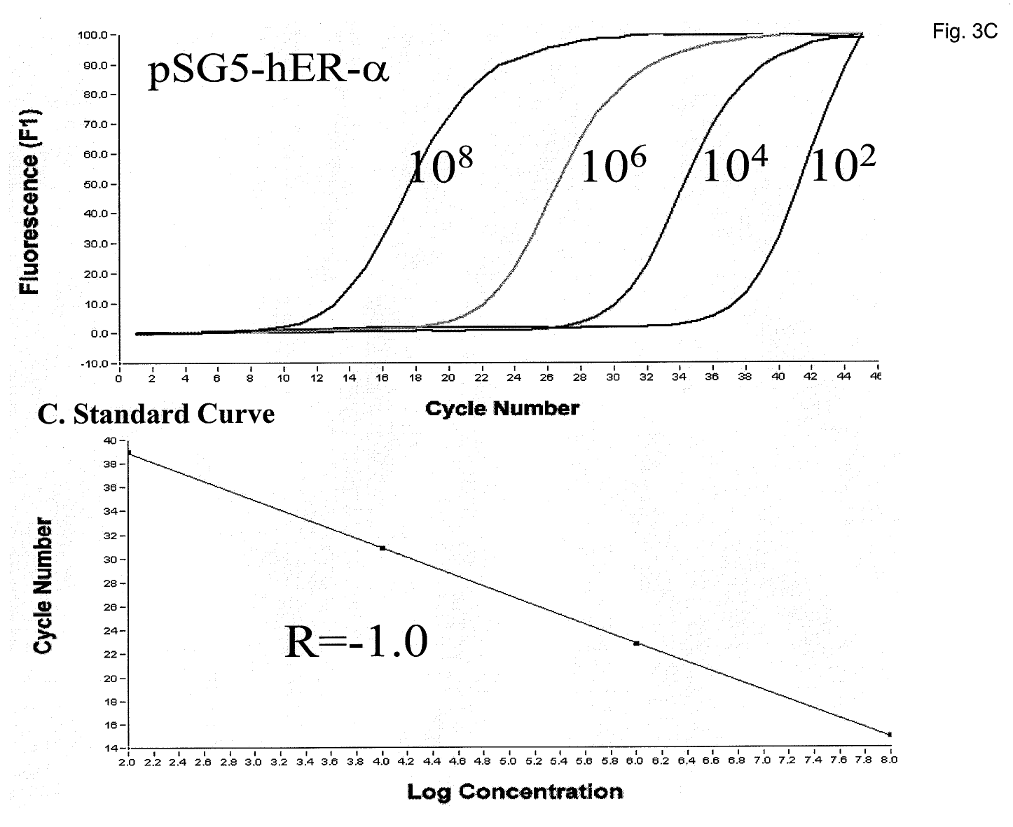

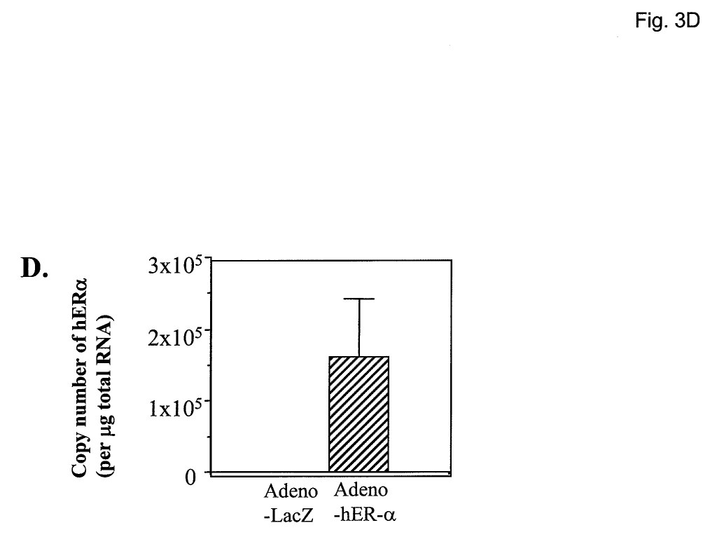

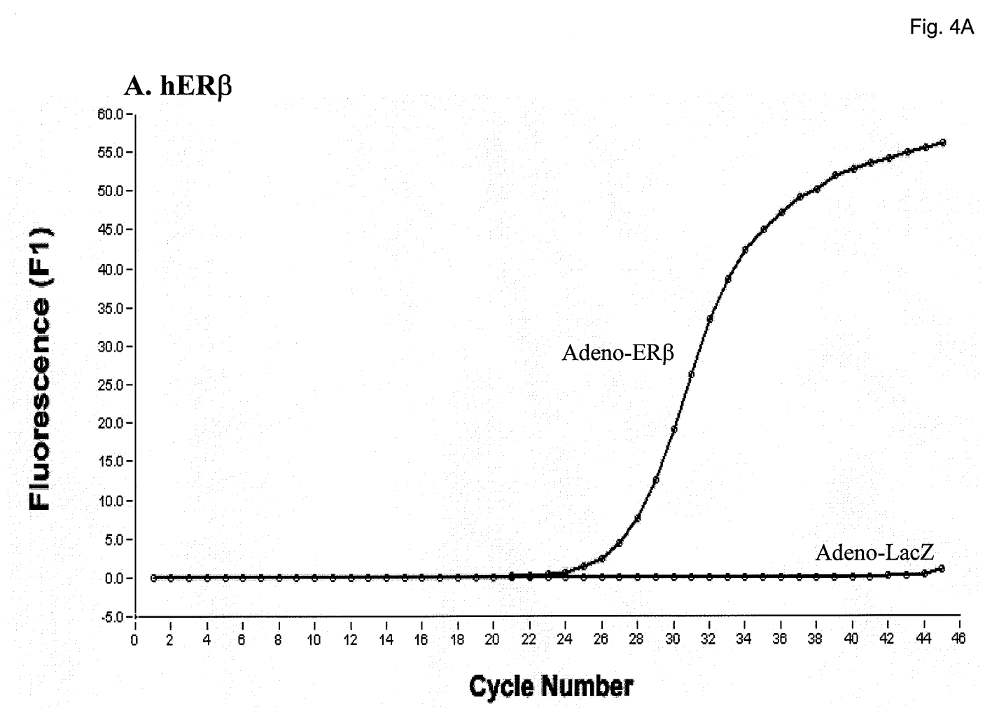

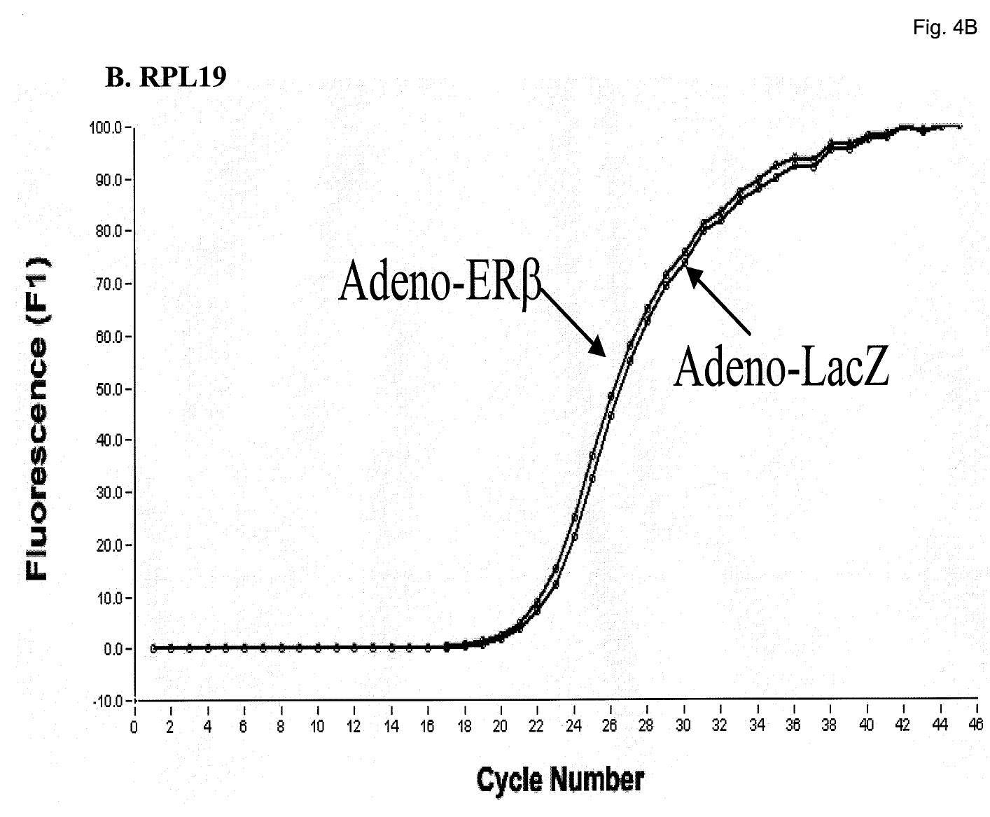

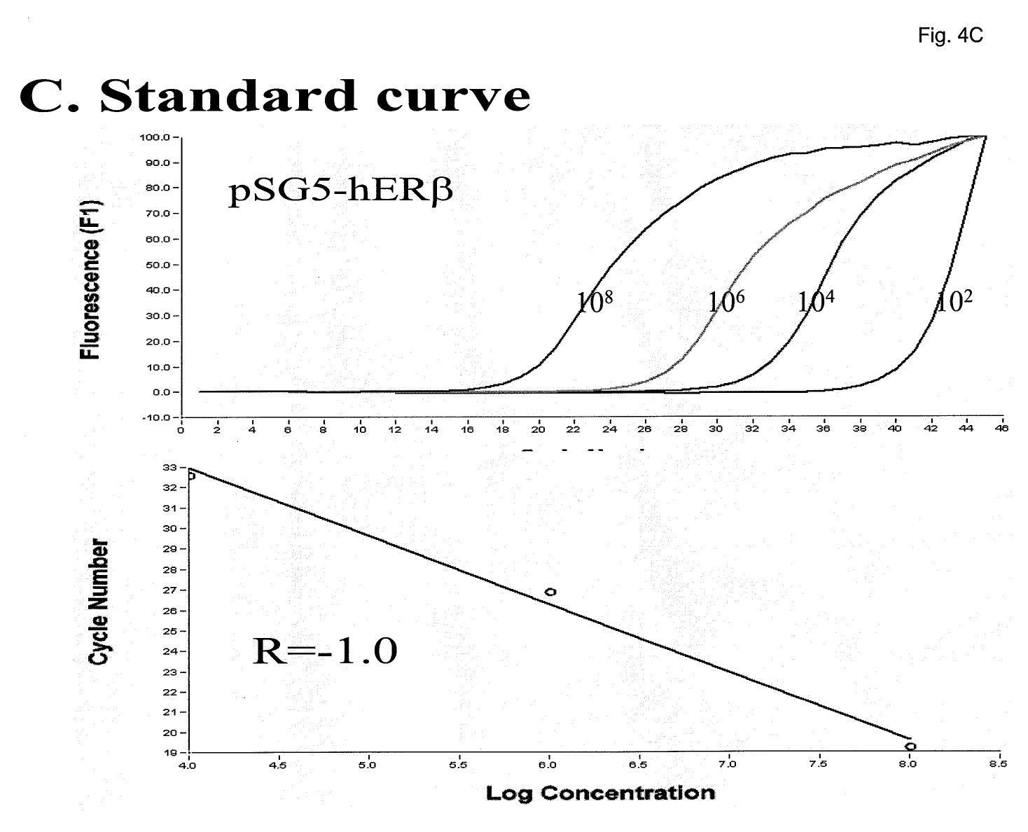

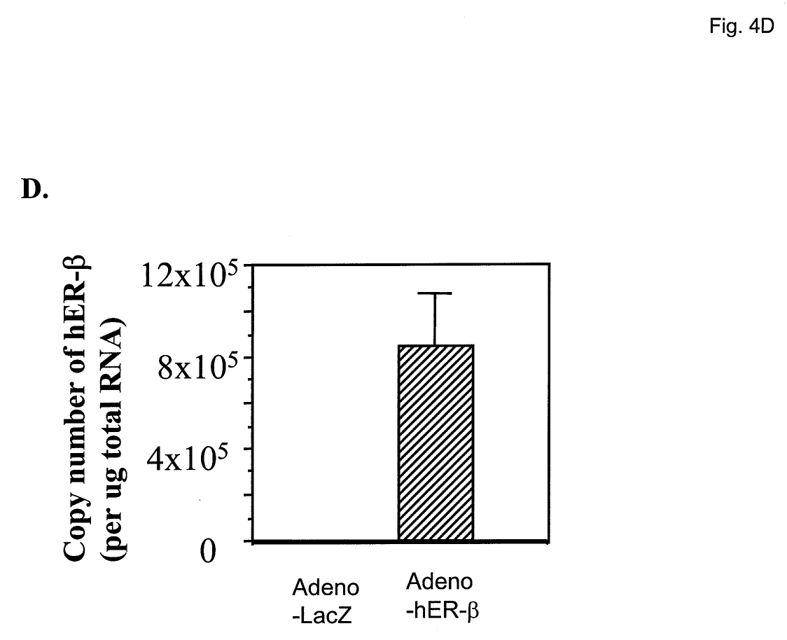

hMSCs were infected with 100 pfu/cell of adeno-ERα, adeno-ERβ or adeno-LacZ (negative control). 48 hours after infection ERα and ERβ mRNA expression was detected by RT-PCR (FIG. 2) and quantitative Real Time RT-PCR (FIGS. 3 and 4). ERα expression was detected in hMSCs infected with adeno-ERα and ERβ expression was detected in hMSCs infected with adeno-ERβ, but not in hMSCs infected with adeno-LacZ (FIG. 3).

To quantify estrogen receptor expression in hMSCs, we used Real Time RT-PCR. hMSCs expressed 1.6×10⁵ copies/μg of total RNA of human ERα in cells infected with 100 pfu/cell Adeno-ERα, compared to 68 copies/μg of total RNA of human ERα in cells infected with 100 pfu/cell adeno-LacZ (FIG. 3). ERβ expression was 8.4×10⁵ copies/μg of total RNA of human ERβ in hMSCs infected with 100 pfu/cell, as compared to 30 copies/μg of total RNA of human ERβ in hMSCs infected with Adeno-LacZ (FIG. 4).

### Example 3

### Regulation of hBMP-6 mRNA Expression by Estrogen in hMSCs Engineered to Express ERα and ERβ

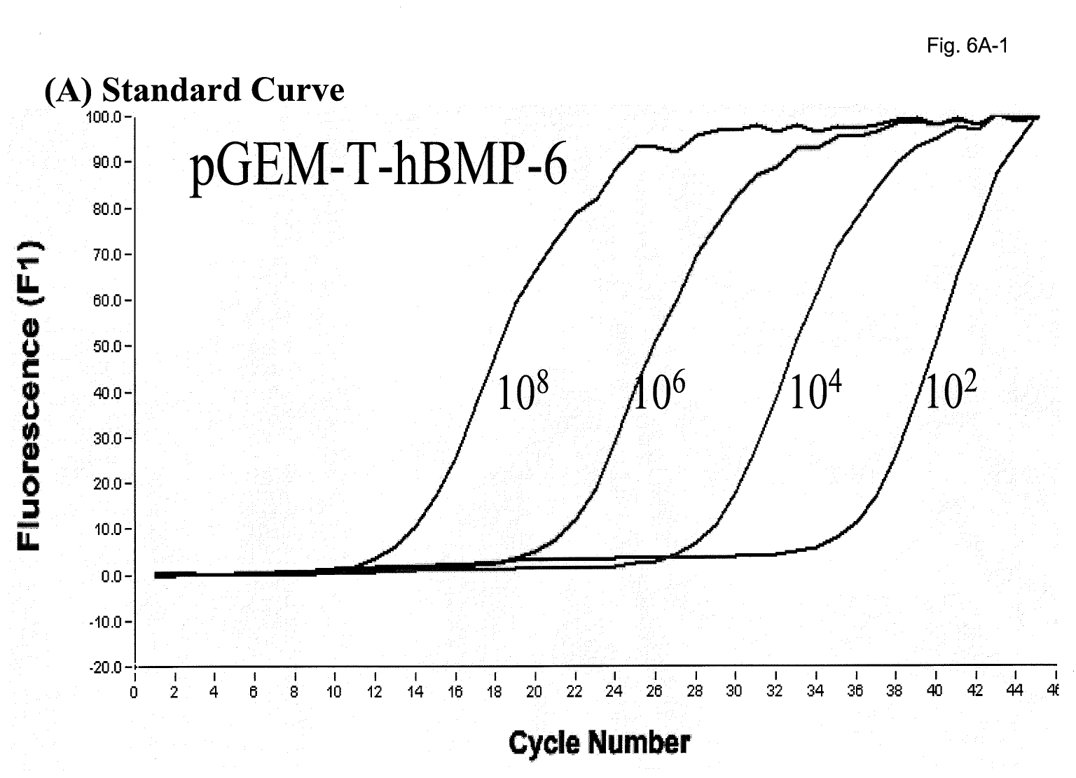

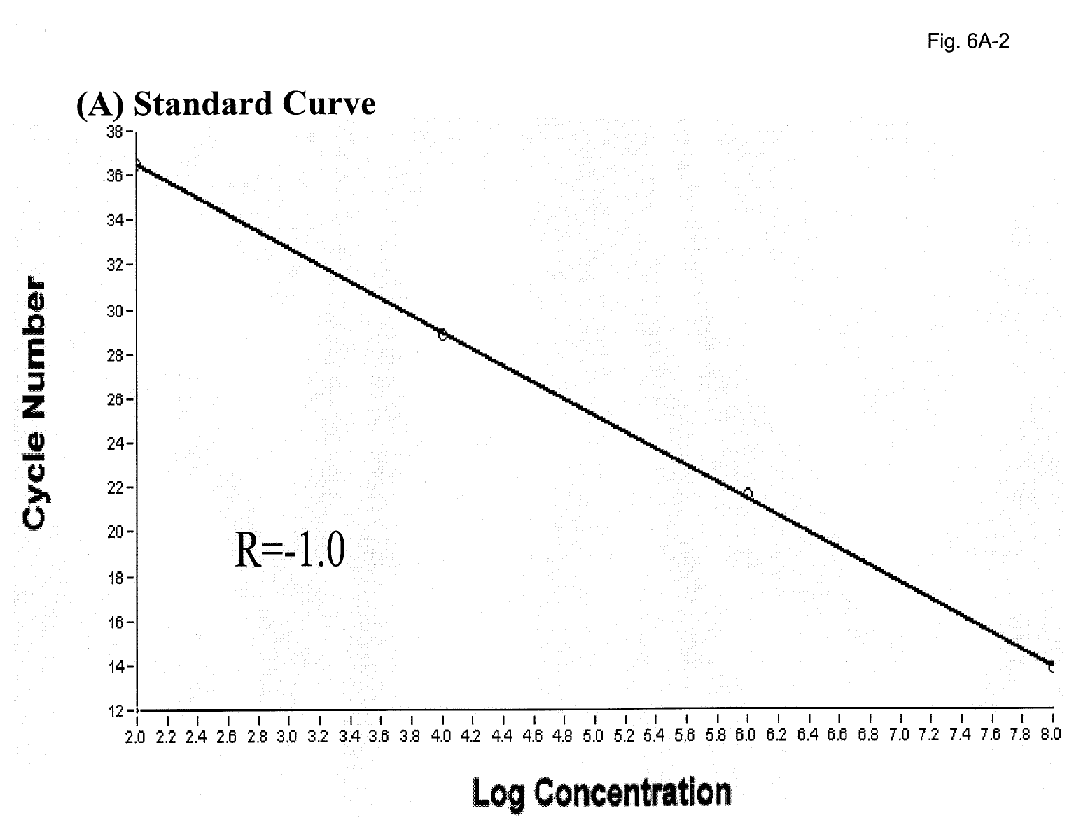

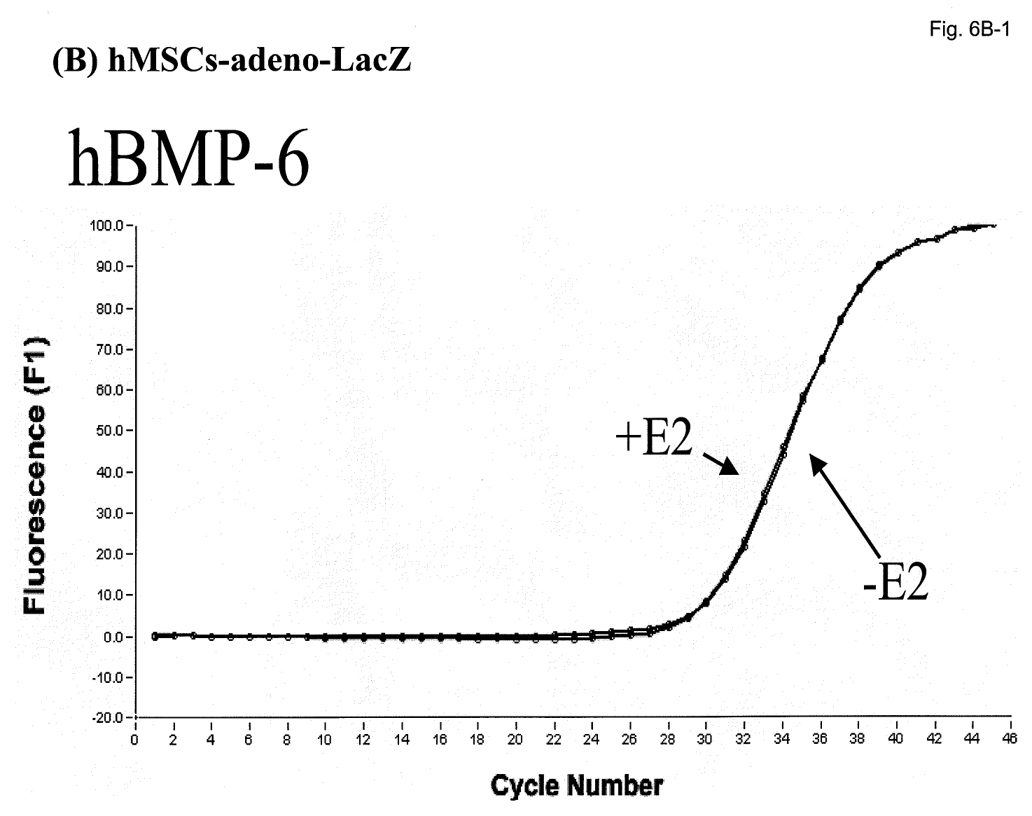

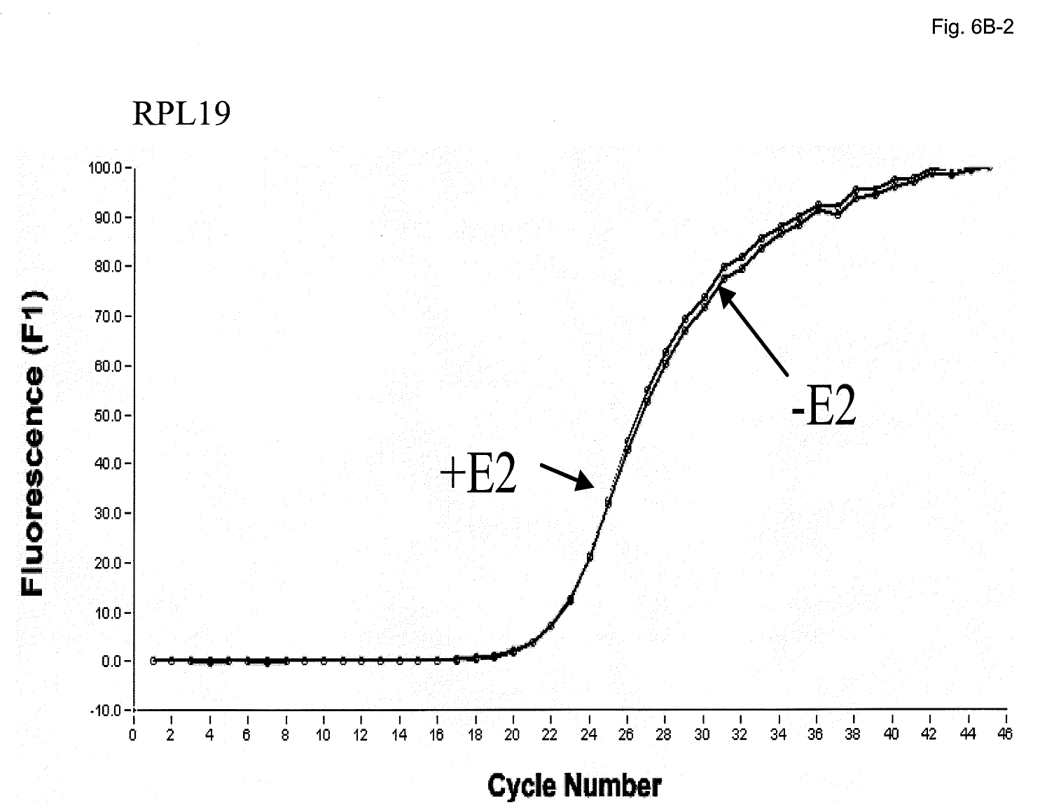

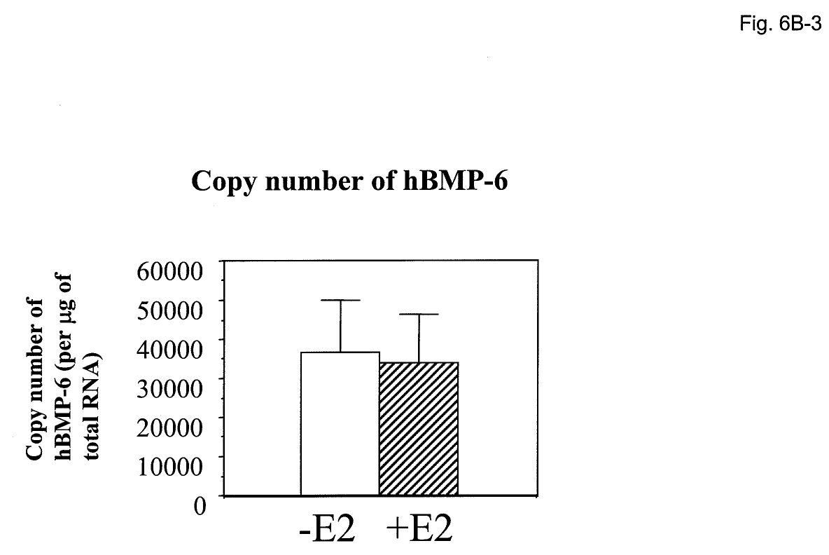

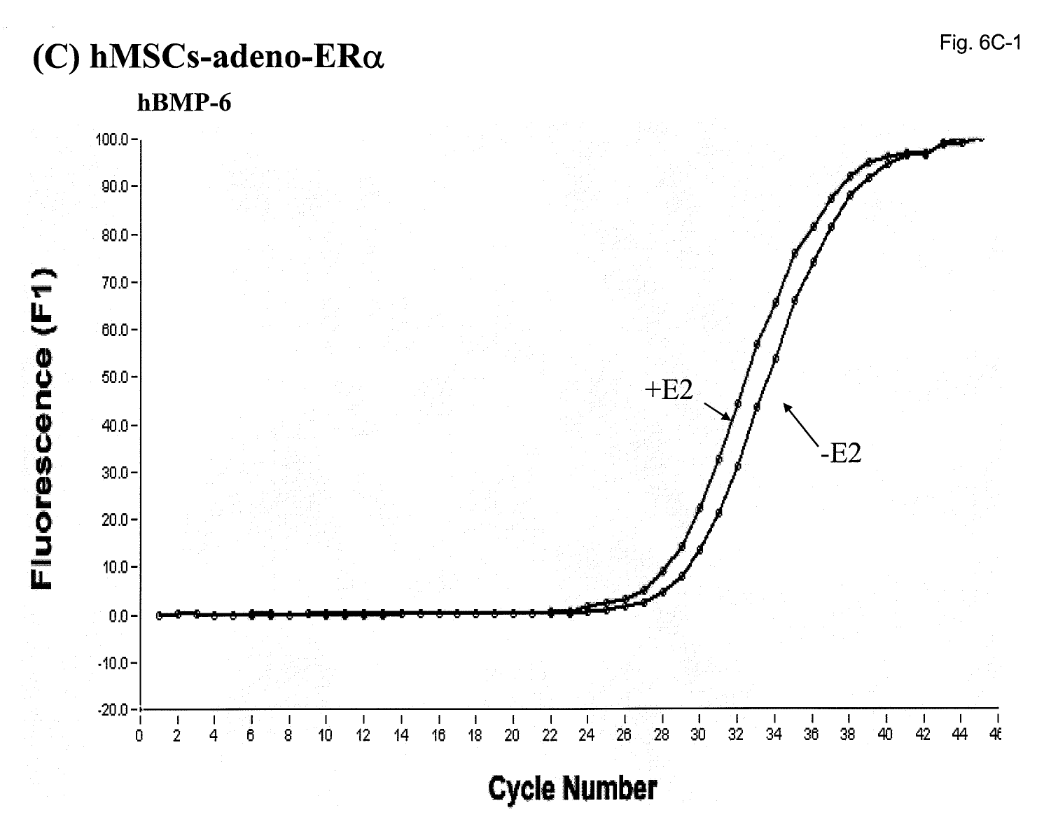

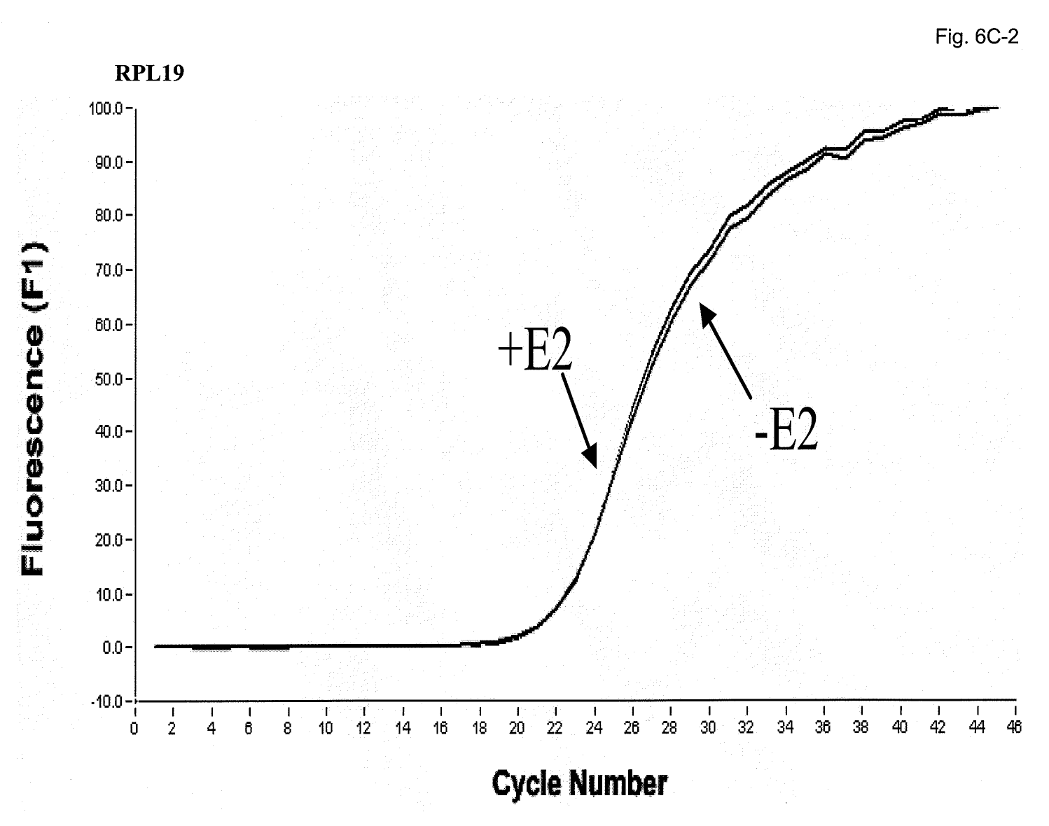

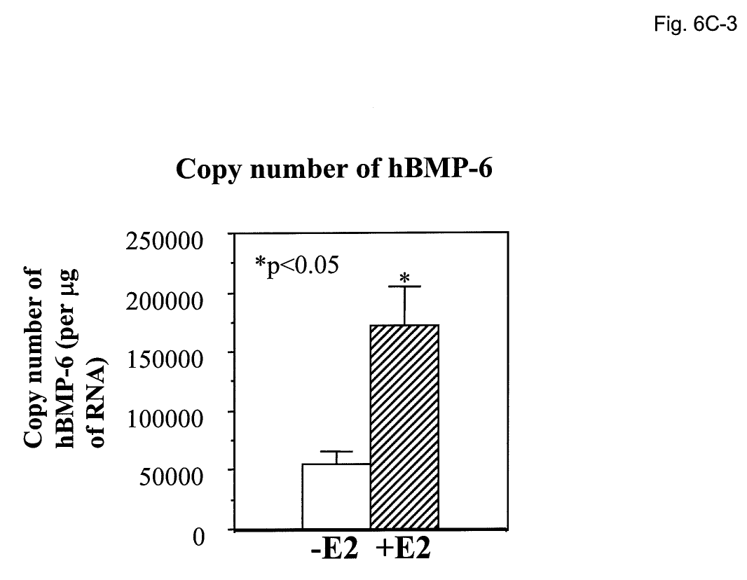

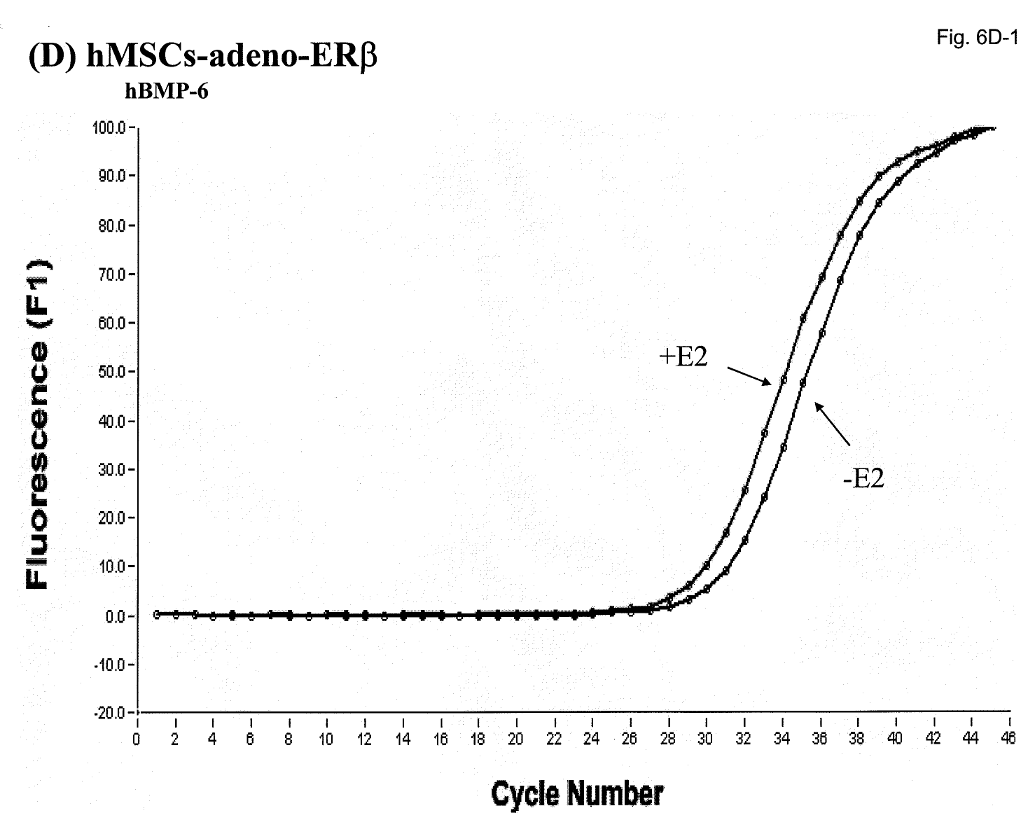

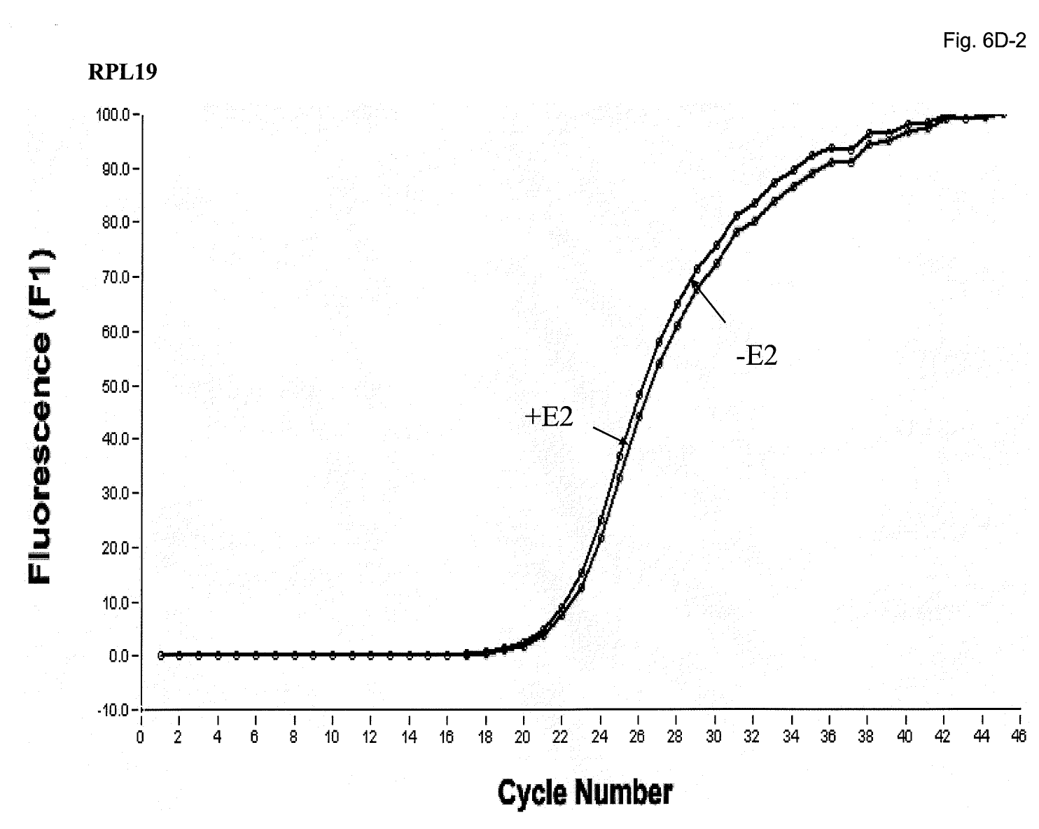

In order to determine downstream effects of estrogen responsiveness on osteogenic factors, hMSC transduced cells were stimulated with 17β-estradiol (E2) (10⁻⁷ M) for 24 hours, following which hBMP-6 mRNA expression was determined. While hMSCs transduced with 100 pfu/cell of either adeno-ERα or adeno-ERβ, produced qualitatively detectable levels of hBMP-6 mRNA, cells transduced with 100 pfu/cell of adeno-LacZ did not (FIG. 5). Quantification of hBMP-6 mRNA expression by Real Time RT-PCR demonstrated that hMSCs transduced with Adeno-ERα, or Adeno-ERβ, and exposed to E2 revealed a 3.2-fold or 2.0-fold increase, respectively, in hBMP-6 mRNA levels, as compared to E2 exposed hMSCs transduced with Adeno-LacZ (FIG. 6).

### Example 4

### hBMP-6 Gene Expression is Regulated by E2 via ERα and ERβ

In order to determine whether estrogen regulates hBMP-6 at the level of gene expression, we examined the effect of E2 on hBMP-6 promoter activity in the mesenchymal stem cell line C3H10T1/2 cells. C3H10T1/2 cells do not express detectable ER levels and require ER transfections/transduction in order to elicit E2-mediated effects on transcription (data not shown).

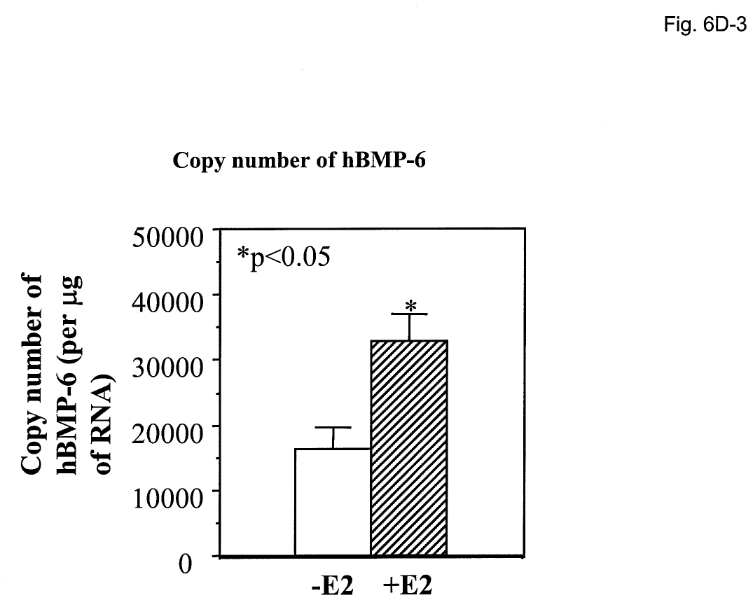

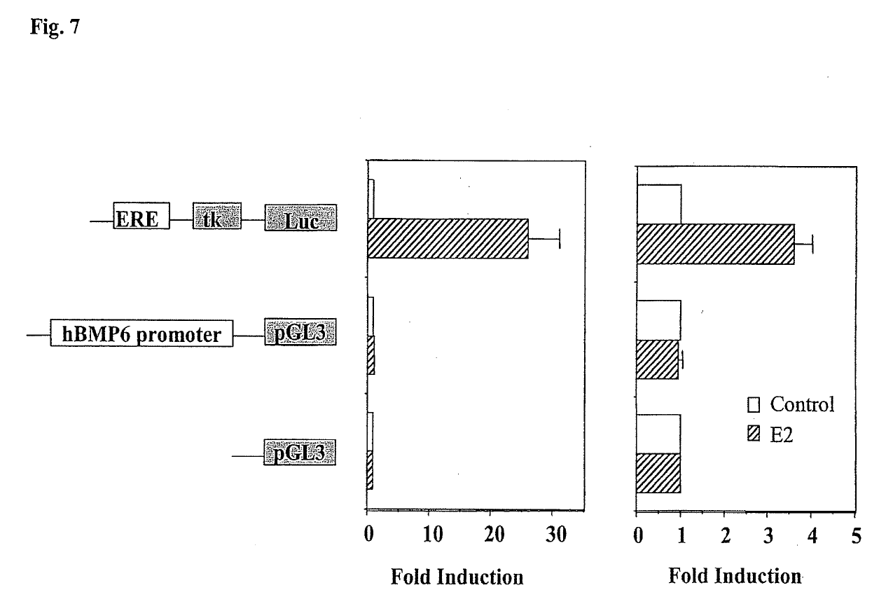

C3H10T1/2 cells have been shown capable of differentiating into osteogenic cells (Gazit et al. 1999; Moutsatsos et al 2001), which suggested their candidacy for evaluating hBMP-6 effects. C3H10T1/2 cells were transiently co-transfected with the ERα or ERβ expression constructs and hBMP-6 promoter-Luc, ERE-tk-Luc (serving as a positive control) or pGL3 (serving as a negative control). Cells were treated with 10⁻⁸ M E2 for 24 hours. Results are shown as fold-increase in luminescence as a function of E2 treatment (FIG. 7), with normalization of background luminescence obtained in pGL3-transfected cells. E2 treatment of cell expressing ERα or ERβ did not result in enhanced luminescence over background levels in C3H10T1/2 cells, although cells expressing the ERE construct revealed significant luminescence when cotransfected with either ERα or ERβ construct.

### Example 5

### hBMP-6 Gene Expression is Regulated at the Transcriptional Level

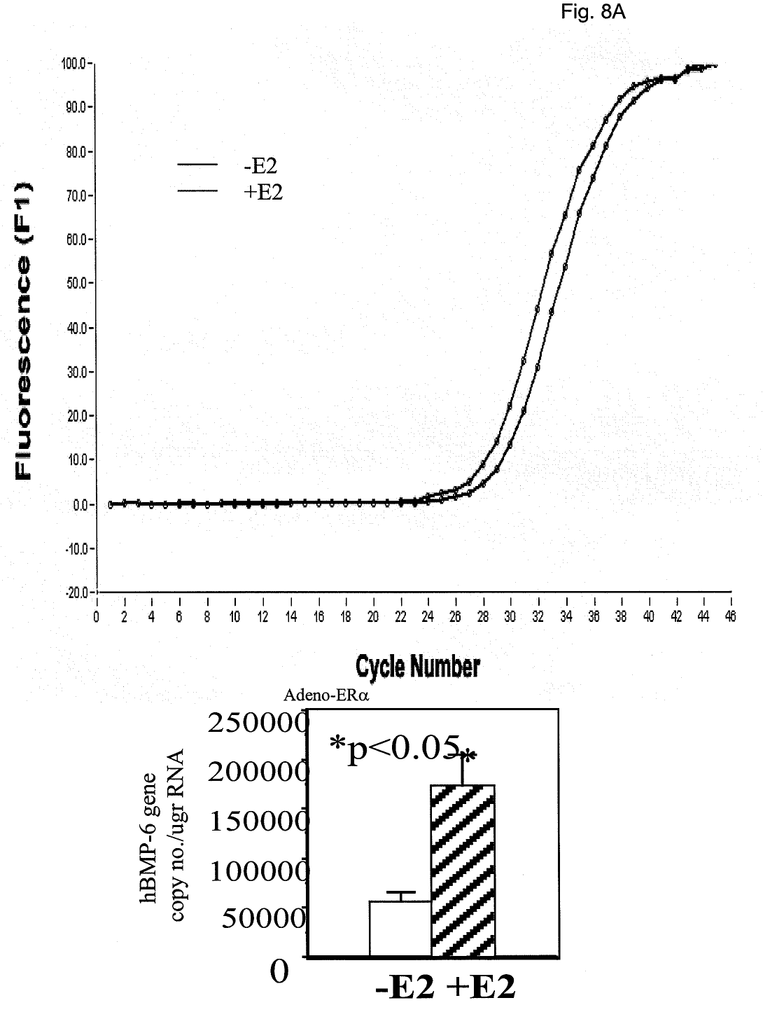

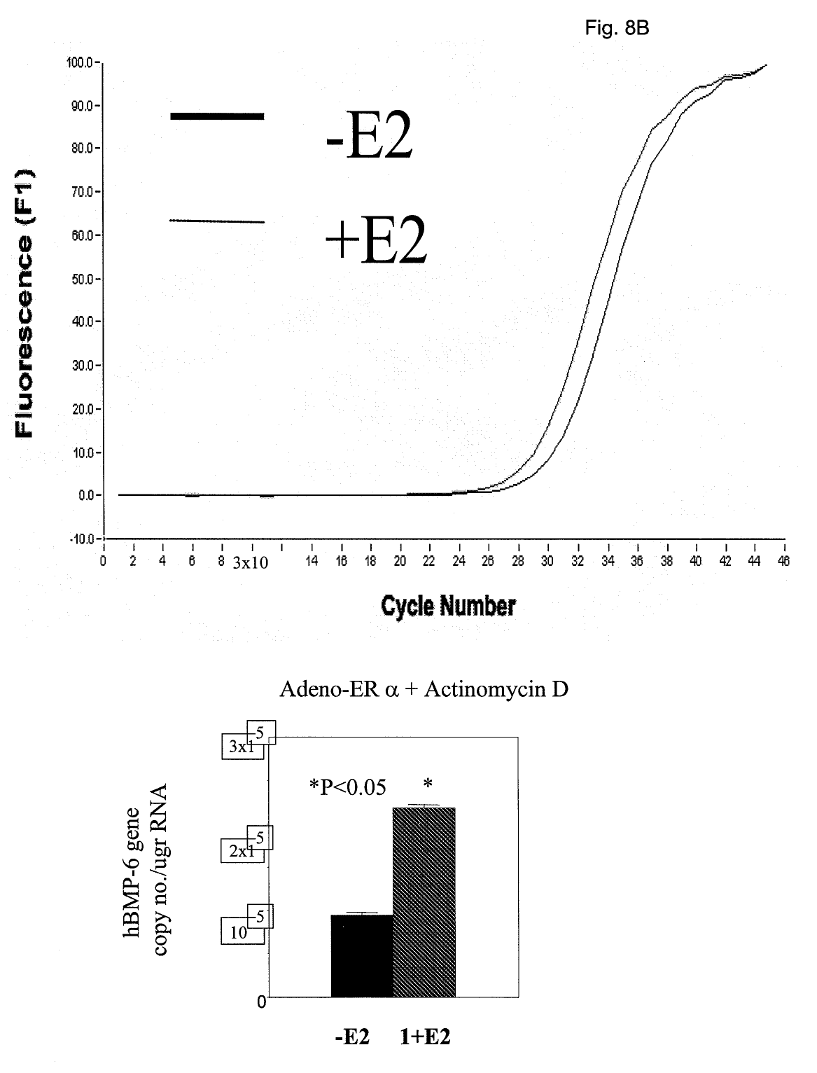

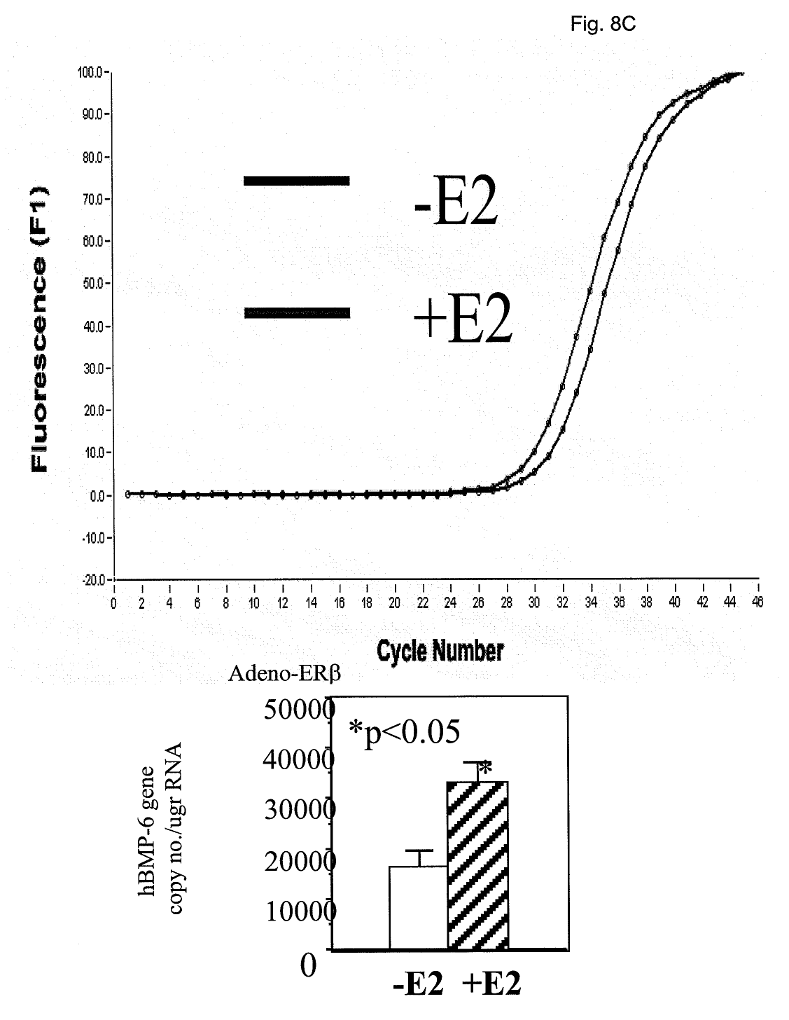

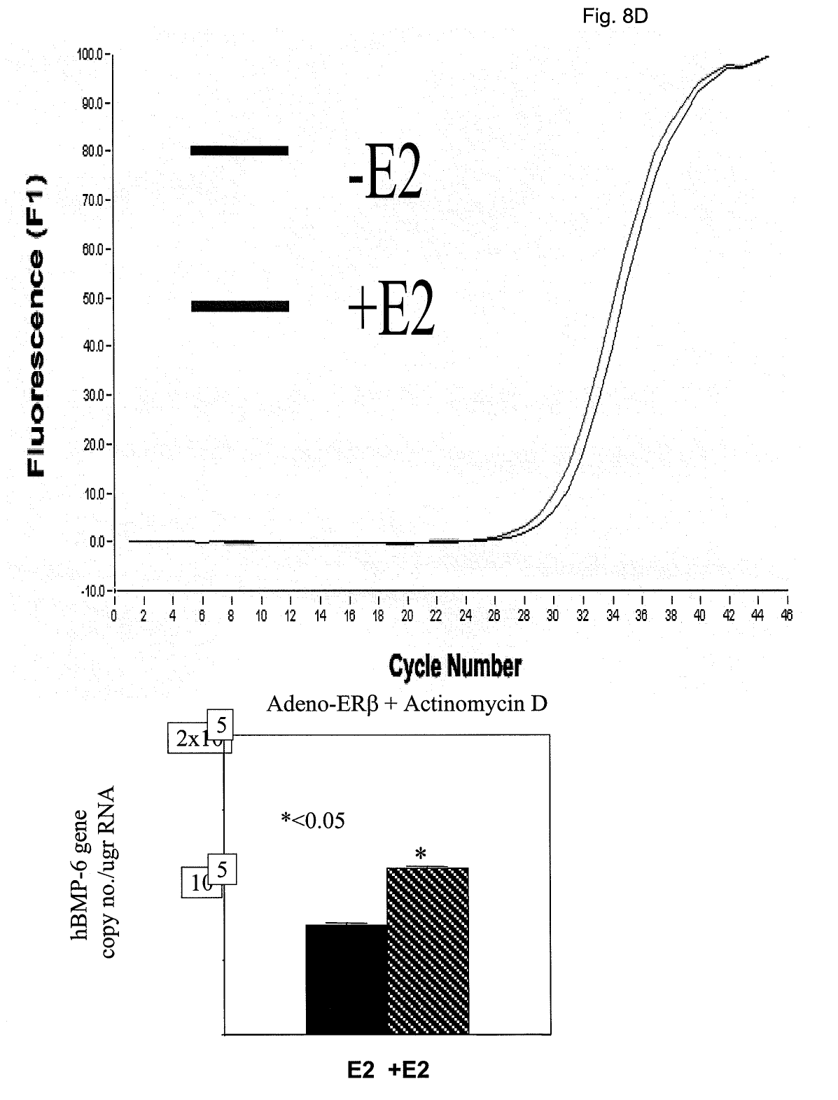

In order to determine whether estrogen regulates hBMP-6 at the level of transcription, we conducted Real-time PCR assays measuring hBMP-6 mRNA expression in cells pulse-chase treated with or without Actinomycin D, which inhibits protein synthesis (FIG. 8).

Cells treated with E2 following transfection with ERα or ERβ demonstrated significantly increased hBMP-6 mRNA expression (p<0.05). There was no statistical difference in the increase in hBMP-6 expression, regardless of whether Actinomycin D was added, indicating the hBMP-6 expression is affected at the transcriptional level.

### Example 6

### hBMP-6 Contains a Previously Unidentified Estrogen Response Element

In order to determine the mechanism of E2-mediated stimulation of hBMP-6 expression, a comprehensive analysis was conducted on the known hBMP-6 gene sequence to establish whether a sequence consistent with an Estrogen Responsive Element (ERE) could be identified. BLAST analysis provided a match for an ERE in the genomic sequence of the hBMP-6 gene, by search query with the following sequence: 5′-nGGnCAnnnTGACCn-3′ (SEQ ID NO:10), where “n” indicates a variable base within the classical ERE sequence.

The ERE canonical sequence is defined by the following polynucleotide string: 5′-AGGTCAnnnTGACCT-3′ (SEQ ID NO:3). Based on BLAST analysis of the human BMP-6 genomic sequence, a sequence consistent with the canonical ERE sequence has been identified, and corresponds to the following: 5′-GGGGCAgtgTGACCA-3′ (SEQ ID NO:1). Similarly, BLAST analysis of the murine BMP-6 gene sequence revealed a match, the sequence corresponding to: 5′-GGGCCActcTGACCC-3′ (SEQ ID NO:2).

The newly-identified hBMP ERE was found to be located in an intron between the first and the second exon of the hBMP-6 gene, at nucleotide coordinates 99,608-99,623.

What is claimed is:

1. A method of regulating expression of BMP-6 in a subject comprising the steps of administering:
(a) a vector comprising a BMP-6 ERE; and
(b) an effective amount of an estrogen or estrogen agonist, thereby regulating expression of BMP-6 in the subject.

2. The method of claim 1, wherein said BMP-6 ERE is at least 90% homologous to SEQ ID Nos. 1 or 2.

3. The method of claim 2, wherein said estrogen is 17β estradiol, 2-hydroxy estradiol, estrone, 2 hydroxy estrone, 16α hydroxy estrone, estriol, ethinyl estradiol, estradiol cypionate, estradiol valerate or estropipate.

4. The method of claim 1, wherein the regulation of BMP-6 expression results in bone repair in the subject.

5. The method of claim 1, wherein the regulation of BMP-6 expression results in the maintenance or increase of bone volume, bone quality, or bone strength in a subject afflicted with osteoporosis.

6. The method of claim 1, wherein the regulation of BMP-6 expression results in osteoblast differentiation in the subject.

7. The method of claim 1, wherein the regulation of BMP-6 expression results in the treatment of a bone disease in the subject.

8. A method of regulating expression of BMP-6 in a subject comprising administering to said subject:
(a) an effective amount of a cell comprising an estrogen receptor or a functional fragment thereof and a vector comprising a BMP-6 ERE operably lined to a BMP-6 gene; and
(b) an effective amount of an estrogen or estrogen agonist;
thereby regulating expression of BMP-6 in the subject.

9. The method of claim 8, wherein the regulation of BMP-6 expression results in bone repair.

10. The method of claim 8, wherein the regulation of BMP-6 expression results in the maintenance or increase of bone volume, bone quality, or bone strength in a subject afflicted with osteoporosis.

11. The method of claim 8, wherein the regulation of BMP-6 expression results in the treatment of a bone disease in the subject.

12. A method of increasing a responsiveness of a cell to an estrogen or estrogen agonist comprising contacting the cell with a vector comprising a BMP-6 ERE, thereby increasing responsiveness of said cell to an estrogen or estrogen agonist.

13. The method of claim 12, wherein said cell further comprises an a or 1 estrogen receptor.

14. A method of enhancing bone repair in a subject in need comprising the steps of:
(a) obtaining a cell from said subject;
(b) transfecting said cell with a vector comprising a BMP-6 ERE operably linked to a BMP-6 gene;
(c) administering the engineered cell to said subject; and
(d) administering to said subject an effective amount of an estrogen or estrogen agonist;
thereby enhancing repair of a bone in a body of said subject in need.

15. The method of claim 14, wherein the enhancement of bone repair results in the maintenance or increase in bone volume, bone quality, or bone strength in a subject afflicted with osteoporosis.

16. A method for the production of transplantable bone matrix, the method comprising the steps of:
(a) obtaining a cell;
(b) transfecting said cell with a vector comprising a BMP-6 ERE operably linked to a BMP-6 gene; and
(c) culturing said cell with cell-associated matrix for a time effective for allowing formation of a transplantable bone matrix;
thereby producing transplantable bone matrix.

17. A method for the identification of a therapeutic agent for the prevention and/or treatment of osteoporosis, comprising:
(a) introducing into an estrogen receptor-expressing cell a vector comprising a BMP-6 ERE operably linked to a BMP-6 gene, further operably lined to a reporter gene;
(b) contacting said cell with a candidate agent; and
(c) monitoring reporter protein expression,
wherein induced reporter protein expression indicates that the candidate agent is a therapeutic agent.


SEQUENCE LISTING (10 sequences)

SEQ ID NO: 1 - DNA, 15 bp, Homo sapiens
ggggcagtgtgacca

SEQ ID NO: 2 - DNA, 15 bp, Mus sp.
gggccactctgaccc

SEQ ID NO: 3 - DNA, 15 bp, Artificial Sequence
  Description of Artificial Sequence Synthetic polynucleotide
aggtcannntgacct

SEQ ID NO: 4 - DNA, 26 bp, Artificial Sequence
  Description of Artificial Sequence Synthetic primer
aagcctcgagtccgctaaacgcctac

SEQ ID NO: 5 - DNA, 25 bp, Artificial Sequence
  Description of Artificial Sequence Synthetic primer
tcagatcttatctctcatggtcgtc

SEQ ID NO: 6 - DNA, 22 bp, Artificial Sequence
  Description of Artificial Sequence Synthetic primer
aaggagactcgctactgtgcag

SEQ ID NO: 7 - DNA, 22 bp, Artificial Sequence
  Description of Artificial Sequence Synthetic primer
atcaggatctctagccaggcac

SEQ ID NO: 8 - DNA, 21 bp, Artificial Sequence
  Description of Artificial Sequence Synthetic primer
ttcccagcaatgtcactaact

SEQ ID NO: 9 - DNA, 19 bp, Artificial Sequence
  Description of Artificial Sequence Synthetic primer
tctctgtctccgcacaagg

SEQ ID NO: 10 - DNA, 15 bp, Artificial Sequence
  Description of Artificial Sequence Synthetic polynucleotide
nggncannntgaccn